(19) United States
(12) Patent Application Publication
(10) Pub. No.: US 2011/0294736 A1
(43) Pub. Date: Dec. 1, 2011

(54) COMPOUNDS AND METHODS FOR THE TREATMENT OF AUTOIMMUNE AND INFLAMMATORY DISEASE

(75) Inventors: Kingston Mills, Dublin (IE); Sarah Higgins, Dublin (IE)

(73) Assignee: THE PROVOST, FELLOWS AND SCHOLARS OF THE HOLY AND UNDIVIDED TRINITY OF QUEEN ELIZABETH NEAR DUBLIN, Dublin (IE)

(21) Appl. No.: 13/141,198

(22) PCT Filed: Dec. 22, 2009
(86) PCT No.: PCT/EP2009/067819

§ 371 Date: Aug. 23, 2011

(30) Foreign Application Priority Data
Dec. 22, 2008 (EP) 08172654.9

(51) Int. Cl. A61K 38/14; A61K 38/06; A61P 17/06; A61P 37/06; A61P 29/00; A61P 1/04

(52) U.S. Cl. 514/13.2; 514/20.9; 514/16.6; 514/18.7

(57) ABSTRACT

The present invention provides a method and composition for the treatment and prevention of an autoimmune disease such a multiple sclerosis which is mediated by autoreactive T cells. The administration of a NOD-1 agonist is shown to mediate an anti-inflammatory immune response. NOD-1 agonists suitable for use in the methods and compositions of the invention include diaminopimelic acid (DAP)-containing muropeptide compounds such as Tri-DAP and M-TriDAP.

### FIELD OF THE INVENTION

The present invention relates to compositions and methods for the treatment and prevention of disease conditions mediated by T-helper 17 (Th17) and/or T-helper 1 (Th1) T lymphocytes (T cells). In particular, the present invention relates to the use of diaminopimelic acid (DAP)-containing muropeptide compounds for the treatment of disease conditions mediated by autoreactive Th17 and Th1 T lymphocytes. The compounds and methods of the invention further have utility in methods for modulating an immune response by suppressing the production of the cytokines Interleukin 17 (IL-17), Interferon gamma (IFN-γ) and Tumour Necrosis Factor alpha (TNF-α), while enhancing the expression of IL-27, IL-10 and IL-35.

### BACKGROUND TO THE INVENTION

Protective immunity against certain diseases is dependent on the differential induction of specific pro-inflammatory T-cell (T lymphocyte) populations by antigen presenting cells (APCs) of the innate immune system, such as dendritic cells (DCs) and macrophages. Two such T-cell populations, responsible for mediating cellular immunity to a wide range of pathogens, are Th1 and Th17 cells. Both Th1, and more recently Th17, T cell populations have been implicated as mediators of autoimmune and chronic inflammatory diseases, and thus serve as relevant cellular targets for immunosuppressive agents. Furthermore, DCs, as initiators of T-cell responses, are therefore a second cellular target for therapies designed to combat inflammatory disease.

Multiple Sclerosis is an inflammatory autoimmune disorder of the central nervous system (CNS), characterised by inflammatory infiltrates of T-cells, B cells, macrophages and focal demyelinating plaques within the CNS. Both Th1 and Th17 cell-mediated responses have been shown to play a role in the development of inflammatory demylelination. Myelin-reactive T-cells from MS patients produce cytokines consistent with a Th1-mediated response, while microarray studies of MS lesions from patients demonstrate increased expression of IL-.

Experimental autoimmune encephalomyelitis (EAE) is an animal model of inflammatory demyelinating disease that shares clinical and neuropathological changes with multiple sclerosis (MS). As such, EAE is a relevant and useful model for dissecting the mechanisms of autoimmune inflammatory responses, and in particular MS. It has been accepted for many years that EAE is largely a CD4⁺ Th1-mediated disease, though a pathogenic role for CD8⁺ T-cells in the induction of EAE has also been demonstrated. More recently however, it has been demonstrated that an IL-17-producing T cell subset plays a critical role in the pathogenesis of EAE. While there is still some debate in the literature, it is likely that Th1 and Th17 cells cooperate to induce the development of organ-specific autoimmunity.

Immature dendritic cells sample antigens (Ag) in the peripheral tissues and, upon antigen capture, mature and migrate to the local lymph nodes (LN), where they present antigen to naïve CD4⁺ T-cells. Naïve T-cells then differentiate and proliferate into specific CD4⁺ effector T-cell populations. The fate of naïve CD4⁺ T-cells is determined, in part, by the cytokine environment which results from cytokine production and output from mature dendritic cells which are present at the site of T-cell receptor (TCR) engagement by the Ag-MHC complex. As such, DCs can exhibit control over the type of T-cell response induced in response to a pathogen.

The cells of the innate immune system, such as dendritic cells, express pathogen recognition receptors (PRRs) that recognise microbial molecular structures and, upon recognition of such structures, activate pro-inflammatory signalling pathways that in turn control the expression of a variety of immune response genes. PRRs can be membrane bound, such as the Toll-like Receptors (TLRs), or cytoplasmically located, such as the nucleotide-binding oligomerization domain (NOD) proteins.

The NOD proteins NOD1 and NOD2 have important roles in innate immunity as intracellular sensors of microbial components derived from peptidoglycan. NOD1 recognises the peptide γ-D-glutamyl-meso-diaminopimelic acid (iE-DAP) and mainly acts as a sensor for gram-negative bacteria, while NOD-2 detects muramyl dipeptide (MDP), found in most bacteria. iE-DAP is the minimal motif recognized by NOD1. L-Ala-γ-D-Glu-mDAP (Tri-DAP) comprises the iE-DAP dipeptide and an L-Ala residue. Similarly to iE-DAP, Tri-DAP is specifically recognized by NOD1.

Consistent with their role as PRRs, NOD ligand binding results in the initiation of pro-inflammatory responses through the activation of the transcription factor, nuclear factor-κB (NF-κB) and members of the mitogen activated protein kinase (MAPK) family. It has been previously shown that Tri-DAP exhibits a 3-fold higher ability to activate NF-κB than iE-DAP.

### SUMMARY OF THE INVENTION

In work leading up to this invention, the inventors have made the surprising discovery that the NOD1 agonist Tri-DAP has immunosuppressive activity and acts to selectively suppress Th1 and Th17 (IL-17 producing T cell) T cell mediated inflammatory autoimmune disease. This observation is entirely unexpected, as previously the in-vitro administration of TriDAP was shown to enhance inflammatory cytokine production. The inventors have now surprisingly identified that the in-vivo administration of TriDAP mediates an anti-inflammatory effect. In particular, expression of the cytokine IL-27 is observed. Without wishing to be bound by theory, the inventors predict that IL-27 is expressed by antigen presenting cells, such as dendritic cells and macrophages. The expression of IL-27 by the antigen presenting cells skews the resulting T cell response away from the expansion of T cells with a Th1 and Th17 phenotype which can in some instances become autoreactive T cells which mediate autoimmune and chronic inflammatory conditions.

Furthermore, the inventors have identified that Tri-DAP is attractive as therapeutic agent, due to its ease of production. Further, due to its low molecular weight, the inventors have identified that TriDAP is unlikely to be significantly immunogenic when administered to humans. The current therapies for the treatment of autoimmune and chronic inflammatory diseases, such as multiple sclerosis, are mainly focused on the use of steroids and other NSAIDs, which are non-specific and which have serious side effects. In particular, certain such treatments primarily act to suppress the expression or functional activity of TNF-alpha. For example, the monoclonal antibody INFLIXIMAB (remicade) targets TNF-alpha function. Although effective in certain patients, such anti-TNF-alpha treatments can be ineffective when treating certain patients, or certain autoimmune conditions, or further, could also result in the occurrence of undesirable side effects. The inventors have therefore identified the utility of the present invention in the treatment of Th1 and/or Th17-mediated diseases and conditions, in particular autoimmune or immune-mediated conditions which occur where aberrant Th1 and/or Th17 responses occur due to the occurrence of autoreactive Th1 and/or Th17 T cells.

According to a first aspect of the present invention there is provided a method of treating or preventing an autoimmune disease which is caused by autoreactive Th1 and/or Th17 T cells, the method comprising the steps of:
providing a therapeutically effective amount of a composition which comprises a diaminopimelic acid (DAP)-containing muropeptide compound, and
administering said composition to a subject in need of such treatment in an amount sufficient to suppress the activation of T-helper 17 lymphocytes (Th17 T cells) and/or a T-helper 1 lymphocytes (Th1 T cells).

In certain embodiments the composition suppresses both a T-helper 17 lymphocyte (Th17) mediated immune response and a T-helper 1 lymphocyte (Th1) mediated immune response.

In certain embodiments, the autoimmune disease which is mediated by the autoreactive T cells is an autoimmune disease or chronic inflammatory diseases. By “autoreactive T cell” it is meant a T cell (T lymphocyte) in particular of the cell lineage Th1 (CD4+ Th 1 helper T cell), or a Th17 (CD4+ T-helper cell) which is specific for a self antigen, a “self antigen” being an antigen expressed by a host, wherein the T cell population of that host would normally be tolerant (i.e. not direct an immune response) to that antigen, under normal homeostatic conditions.

In certain embodiments, the autoimmune disease can be selected from the group consisting of, but not limited to: multiple sclerosis (MS), rheumatoid arthritis (RA), inflammatory bowel disease (IBD) including Crohn's disease and ulcerative colitis, type 1 diabetes and psoriasis.

In certain embodiments, the diaminopimelic acid (DAP)-containing muropeptide compound is Tri-DAP. Tri-DAP may also be represented as TriDAP, Tri_{DAP}, L-Ala-γ-D-Glu-mDAP or _{L}-Ala-_{D}-Glu-mesoDAP). TriDAP may also be referred to as L-alanyl-γ-D-glutamyl-meso-diaminopimelic acid. TriDAP is a tri-peptide comprising the iE-DAP dipeptide (γ-D-glutamyl-meso-diaminopimelic acid) and an L-Ala (alanine) residue. Tri-DAP has the chemical formula C16H26N4O8. It has a molecular weight of around 390.39 kDa. As herein defined “mesoDAP” relates to meso-diaminopimelate. The term “diaminopimelyl” refers to the incorporation of mesoDAP into the peptide chain.

Tri-DAP has the molecular structure shown in Formula 1a below:

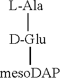

Tri-DAP has the chemical structure as shown below in Formula 1b:

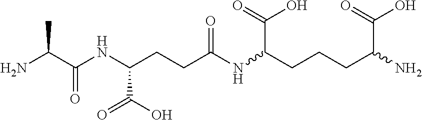

In certain further embodiments, the diaminopimelic acid (DAP)-containing muropeptide compound can be M-TriDAP (MurNAc-L-Ala-γ-D-Glu-mDAP), which is also called DAP-containing muramyl tripeptide (muratripeptide), this being a peptidoglygan (PGN) degradation product which can be found in gram negative bacteria.

M-TriDAP has the molecular structure shown in Formula II below:

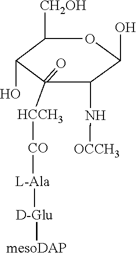

In certain further embodiments where the diaminopimelic acid (DAP)-containing muropeptide compound comprises a muropeptide, the muropeptide may be a murotetrapeptide, for example, GM-TRI_{DAP} (GlcNAc-MurNAc tripeptide muropeptide).

In certain further embodiments the murotetrapeptide may be M-Tetra_{DAP} of formula III:

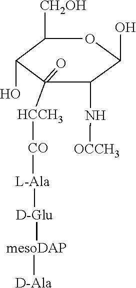

In certain embodiments, synthetic analogues may be provided based on the diaminopimelic acid (DAP)-containing muropeptide compounds of the invention. Furthermore, in certain embodiments, peptidomimetics of the compounds for use in the invention may be prepared, as and where appropriate, as described hereinafter.

In certain embodiments, the method of this aspect of the invention can further comprise the step of administering at least one Toll-like Receptor agonist to the subject. The Toll-like receptor (TLR) agonist may be administered before, along with (simultaneously) or after (sequentially) the administration of the administration of the composition of this aspect of the invention.

In certain embodiments, the TLR agonist is a pharmaceutically acceptable TLR agonist. The TLR agonist may be specific to any defined human Toll-like receptor. In specific embodiments, the TLR agonist is a ligand for at least one of TLR2, TLR4 or TLR9. In certain embodiments, the TLR agonist can be capable of inducing IL-27 production by dendritic cells. In further embodiments the TLR agonist may be selected from any one or more of LPS (lipopolysaccharide), CpG motifs including CpG-containing oligodeoxynucleotides (CpG ODN), dsRNA, Poly (I:C) and Pam-3Cys.

A further aspect of the present invention provides a pharmaceutical composition for use in the treatment and/or prevention of an autoimmune disease which is mediated by autoreactive Th1 and/or Th17 T cells, the composition comprising a diaminopimelic acid (DAP)-containing muropeptide compound along with at least one pharmaceutical excipient, diluent or carrier which can be selected depending on the intended route of administration.

In various further aspects of the invention, the compositions and methods of the invention can be used to suppress or inhibit the production of at least one of the cytokines chosen from the group comprising of: Interleukin 17 (IL-17), Interferon gamma (IFN-γ) and Tumour Necrosis Factor alpha (TNF-α). In certain embodiments, the compositions and methods of the invention may further enhance the production of at least one cytokine selected from IL-27, IL-10 and IL-35.

Cytokines such as interleukin 17, interferon gamma or tumour necrosis factor have defined roles in the development of a number of disease conditions and in particular autoimmune conditions. Accordingly, in various further aspects, the invention extends to administering TriDAP, or related compounds to a subject in a therapeutically effective amount in order to treat, prevent or ameliorate the disease condition.

A still further aspect of the present invention provides a diaminopimelic acid (DAP)-containing muropeptide for use in the treatment or the prevention of an autoimmune disease or a chronic inflammatory disease which is mediated by autoreactive Th1 and/or Th17 T cells.

In certain embodiments the diaminopimelic acid (DAP)-containing muropeptide is TriDAP. In certain embodiments the autoimmune disease is selected from: multiple sclerosis (MS), rheumatoid arthritis (RA) and type 1 diabetes wherein the chronic inflammatory disease is selected from inflammatory bowel disease (IBD), Crohn's disease, ulcerative colitis and psoriasis.

In certain embodiments the use further comprises administering at least one Toll-like Receptor agonist to the subject. The at least one Toll-like Receptor agonist can be an agonist for at least one of Toll-like Receptor 2, Toll-like Receptor 4 or Toll-like Receptor 9. The Toll-like Receptor agonist can be selected from the group consisting of: LPS (lipopolysaccharide), CpG motifs, CpG-containing oligodeoxynucleotides (CpG ODN), dsRNA, Poly (I:C) and Pam-3Cys.

The composition may further comprise at least one ERK protein kinase inhibitor, which can be PD98059 or U0126.

A yet further aspect of the present invention provides for the use of a diaminopimelic acid (DAP)-containing muropeptide in the preparation of a medicament for the treatment of an autoimmune disease or a chronic inflammatory disease which is mediated by autoreactive Th1 and/or Th17 T cells.

In certain embodiments, the medicament may further comprise or be administered along with at least one Toll-like Receptor agonist (a TLR agonist). The TLR agonist may be specific to any defined human Toll-like receptor. In specific embodiments, the TLR agonist has specificity for TLR2, TLR4 or TLR9. In certain embodiments, the TLR agonist can be capable of inducing IL-27 production by DCs. In further embodiments the TLR agonist may be selected from any one or more of LPS (lipopolysaccharide), CpG motifs including CpG-containing oligodeoxynucleotides (CpG ODN), dsRNA, Poly (I:C) and Pam-3Cys. Without wishing to be bound by theory, the inventors have observed, based on the experimentation described herein, that the administration of TriDAP can cause IL-27 production from dendritic cells. However, the co-administration of both IL-27 and at least one TLR agonist is observed to work in a synergistic manner to substantially enhance the production of IL-27 by antigen presenting cells such as dendritic cells.

In certain embodiments, the medicament may further comprise or be administered along with at least one ERK (extracellular signal regulated kinase) inhibitor. Without wishing to be bound by theory, the inventors predict that the further administration of at least one ERK inhibitor as part of, or contemporaneously with the compositions of the invention as hereinbefore defined can upregulate IL-27 cytokine production. It has further been observed that the administration of an ERK inhibitor can attenuate EAE by suppressing IL-23 and IL-1 cytokine production by dendritic cells. The administration of at least one ERK inhibitor resulted in upregulation of IL-27, irrespective of whether a composition of the invention was administered along with a TLR agonist as described hereinbefore. In certain embodiments, the ERK inhibitor is an inhibitor of the ERK protein kinase and may be selected from the group comprising, but not limited to: PD98059 or U0126.

A yet further aspect of the present invention provides a composition comprising a diaminopimelic acid (DAP)-containing muropeptide for use in treating or preventing an autoimmune disease or a chronic inflammatory disease.

In certain embodiments, the composition may further comprise at least one Toll-like Receptor agonist. The TLR agonist may be specific to any defined human Toll-like receptor. In specific embodiments, the TLR agonist has specificity for TLR2, TLR4 or TLR9. In certain embodiments, the TLR agonist can be capable of inducing IL-27 production by DCs. In further embodiments the TLR agonist may be selected from any one or more of LPS (lipopolysaccharide), CpG motifs including CpG-containing oligodeoxynucleotides (CpG ODN), dsRNA, Poly (I:C) and Pam-3Cys.

In various further aspects of the invention, there is provided combined medicaments comprising the compounds of the invention along with at least one compound selected from the groups consisting of: a steroid, a non-steroidal anti-inflammatory drug (NSAID) or a cytokine inhibitor. Such combined medicaments may be used in the methods of the invention for the treatment of autoimmune diseases or chronic inflammatory diseases.

In a yet further aspect of the present invention there is provided a method of treating an autoimmune disease, wherein the method comprises modulating a function (e.g., modulating one or more biological activities of NOD-1) in a NOD-1 expressing or responsive cell and/or tissue (e.g., an antigen presenting cell, in particular a dendritic cell). The method includes contacting the NOD-1-responsive cell and/or NOD-1-responsive tissue with a NOD-1 modulator, e.g., a NOD-1 binding agent, (e.g. a composition comprising a diaminopimelic acid (DAP)-containing muropeptide), in an amount sufficient to modulate the function of the NOD-1-responsive cell or tissue (or the biological activity of NOD-1 in the cell or tissue) such that a Th1 and/or Th17 mediated immune response is suppressed, that is the activation of T cells having a Th1 or Th17 phenotype, or the differentiation of a naïve CD4+ T cell into a Th1 or Th17 T cell is reduced or inhibited. In one embodiment, the contacting step can be effected in vitro, e.g., in a cell lysate or in a reconstituted system. Alternatively, the subject method can be performed on cells in culture, e.g., in vitro or ex vivo. For example, cells (e.g., purified or recombinant cells) can be cultured in vitro and the contacting step can be effected by adding the NOD-1 modulator to the culture medium. Typically, the NOD-1-responsive cell is a mammalian cell, e.g., a human cell. In some embodiments, the NOD-1-responsive cell is an antigen presenting cell, such as a dendritic cell, or a cellular population associated therewith, such as a precursor cell. In other embodiments, the method can be performed on cells present in a subject, e.g., as part of an in vivo protocol, or in an animal subject (including, e.g., a human subject, or an in vivo animal model. The in vivo protocol can be therapeutic or prophylactic, and the inflammatory model can be, for example, an EAE model, or a genetically modified model (e.g., an animal model having over expressed NOD-1, or a NOD-1 mutation or deletion). For in vivo methods, the NOD-1 modulator, alone or in combination with another agent, can be administered to a subject suffering from an autoimmune disease such as multiple sclerosis, rheumatoid arthritis, or psoriasis in an amount sufficient to modulate, one or more NOD-1 mediated activities or functions in the subject, typically a pro-inflammatory immune response, and most particularly a pre-inflammatory immune response mediated by Th1 cells (also known as Th1 T lymphocytes) and/or Th17 T lymphocytes which could result in the onset and progression of a chronic inflammatory disease or an autoimmune disease, such as multiple sclerosis. In some embodiments, the amount or dosage of the NOD-1 modulator that is administered can be determined prior to administration by testing in vitro or ex vivo, the amount of NOD-1 modulator required to alter, e.g., decrease or inhibit, one or more of NOD-1 activities (e.g., one or more NOD-1 biological activities described herein). Optionally, the in vivo method can include the step(s) of identifying (e.g., evaluating, diagnosing, screening, and/or selecting) a subject having, or at risk of having, one or more symptoms associated with the autoimmune disorder or condition.

In certain further aspects of the invention there is provided the use of a NOD-1 modulator, in particular a NOD-1 agonist compound which suppresses a pro-inflammatory immune response mediated by Th1 T lymphocyte cells and/or Th17 T lymphocyte cells in the preparation of a medicament for the treatment of an autoimmune disease or a chronic inflammatory disease.

A yet further aspect of the present invention provides a NOD-1 modulator compound which suppresses a pro-inflammatory immune response mediated by Th1 T lymphocyte cells and/or Th17 T lymphocyte cells for use in treating an autoimmune disease or a chronic inflammatory disease which is mediated by autoreactive Th1 or Th17 T cells.

In certain embodiments the NOD-1 modulator comprises the peptide γ-D-glutamyl-meso-diaminopimelic acid (iE-DAP), or Tri-DAP, or M-TriDAP or an analogue, derivative or peptidomimetic thereof.

A yet further aspect of the invention provides the use of a NOD-1 modulator to enhance IL-27 production in the preparation of a medicament for the treatment of an autoimmune disease by inhibiting IL-23 and IL-17 cytokine levels and/or production. In particular, the production of IL-23 and/or IL-17 by antigen presenting cells, such as dendritic cells, or T cells is suppressed.

A yet further aspect of the present invention provides a composition comprising a diaminopimelic acid (DAP)-containing muropeptide for use in administering to subjects having an autoimmune disease which is mediated by autoreactive T cells of the phenotype Th1 or Th17 in order to inhibit or down-regulate the production of at least one of the Th1 and Th17 associated cytokines IFN-γ, TNF-α, and IL-17.

In various further aspects, the invention provides kits for carrying out the therapeutic regimens of the invention. Such kits may comprise, in one or more containers, therapeutically or prophylactically effective amounts of the compositions of the invention in a pharmaceutically acceptable form. Such kits may further include instructions for the use of the compositions of the invention, or for the performance of the methods of the invention, or may provide further information to provide a physician with information appropriate to treating a chronic inflammatory or autoimmune disease.

A yet further aspect of the present invention provides the use of a composition comprising or consisting of TriDAP or an analog thereof to reduce the production of IL-17 for the treatment of an autoimmune disease or chronic inflammatory condition. In certain embodiments, the composition further comprises at least one Toll-like Receptor agonist. The composition may further enhance IL-27 cytokine levels, and may also reduce at least one of TNF-alpha and IFN-gamma levels, and may further enhance IL-10 levels. The modulation of cytokine levels can be measured by the skilled person using techniques which are well known to the person skilled in the art, such as an ELISA test to quantify the presence and the amount of a cytokine in a sample, such as a cellular supernatant.

In various further aspects, the present invention extends to a method, combination, agent, use, system of kit of parts according to any of the foregoing aspects or embodiment wherein the patient is human.

In a still further aspect, the present invention extends to any novel method of treating an autoimmune condition or chronic inflammatory disease which is caused by autoreactive Th1 and/or Th17 cells, or from an immune reaction mediated by IL-17 in a patient as herein described.

### BRIEF DESCRIPTION OF THE FIGURES

FIG. 1 shows that in vivo administration of the NOD1 agonist Tri-DAP attenuates experimental autoimmune encephalomyelitis (EAE).

FIG. 2 shows that the NOD1 agonist Tri-DAP attenuates experimental autoimmune encephalomyelitis (EAE).

FIG. 3 shows that the NOD1 agonist Tri-DAP suppresses MOG-specific inflammatory cytokines during EAE induced by immunization with MOG and CFA.

FIG. 4 shows that treatment with the NOD1 agonist Tri-DAP suppresses IL-17-expressing and IFN-γ-expressing CD3⁺ T-cells in the brains of mice with EAE.

FIG. 5 shows that treatment with the NOD1 agonist Tri-DAP suppresses IL-17-expressing and IFNγ-expressing CD3⁺ CD4⁺ T cells in the brains of mice with EAE.

FIG. 6 shows that treatment with the NOD1 agonist Tri-DAP suppresses IL-17-expressing and IFN-γ-expressing CD3⁺ CD4⁻ T cells in the brains mice with EAE.

FIG. 7 shows that treatment with the NOD1 agonist Tri-DAP enhances EBI3 mRNA expression in the inguinal lymph nodes of mice 72 hours post EAE induction.

FIG. 8 (a) shows that treatment with the NOD-1 agonist Tri-DAP enhances gene expression of LPS induced EBI3 in DCs.

FIG. 8 (b) shows that treatment with the NOD-1 agonist Tri-DAP enhances gene expression of LPS induced IL-27p28 in DCs.

FIG. 9 (a) shows that inhibition of ERK enhances Tri-DAP synergy with LPS for gene expression of EBI3 in DC.

FIG. 9 (b) shows that inhibition of ERK enhances Tri-DAP synergy with LPS for gene expression of IL-27p28 in DCs.

FIG. 10 (a) shows that enhanced LPS induced IL-12p40 and IL-12p70 production by DCs following treatment with Tri-DAP is dependent on p38 activation.

FIG. 10 (b) shows that enhanced LPS induced IL-23 and IL-10 production by DCs following treatment with Tri-DAP is dependent on p38 activation.

FIG. 11 (a) shows that enhanced CpG induced IL-12p40 and IL-12p70 production by DCs following treatment with Tri-DAP is dependent on p38 activation.

FIG. 11 (b) shows that enhanced CpG induced IL-23 and IL-10 production by DC following treatment with Tri-DAP is dependent on p38 activation.

FIG. 12 (a) shows that enhanced LPS induced IL-12p40 and IL-12p70 production by DC following treatment with Tri-DAP is partially dependent on ERK activation.

FIG. 12 (b) shows that enhanced LPS induced IL-23 and IL-10 production by DC following treatment with Tri-DAP is partially dependent on ERK activation.

FIG. 13 (a) shows that enhanced CpG induced IL-12p40 and IL-12p70 production by DC following treatment with Tri-DAP is partially dependent on ERK activation.

FIG. 13 (b) shows that enhanced CpG induced IL-23 and IL-10 production by DC following treatment with Tri-DAP is partially dependent on ERK activation.

FIG. 14 shows the clinical score of SJL mice treated with the Nod-1 agonist Tri-DAP.

FIG. 15A shows IFN-gamma and IL-17 expression in spleen cells. FIG. 15B shows IL-17 production in the lymph node.

FIG. 16A shows IL-10 expression in the supernatants of peritoneal exudates cells from mice treated with and without TriDAP, while FIG. 16B shows TGF-beta expression.

FIG. 17 shows 4 graphs representing the expression levels of the cytokines IL-10, IL-27 and TGF-β and of the chemokine MIP-1α in the supernatants of TLR agonist-activated spleen cells from mice treated with PBS or TriDAP. Expression levels of IL-10 and IL-27 are seen to be enhanced in the spleen cells from TriDAP-treated mice following in vitro stimulation with a TLR agonist.

FIG. 18 shows that interferon gamma production by T cells is lower when stimulated with antigen presenting cells obtained from TriDAP treated mice when compared with antigen presenting cells obtained from control mice.

FIG. 19 shows a graph showing IL-17 expression levels by antigen-specific T cells stimulated with dendritic cells pulsed with antigen and LPS with and without TriDAP.

FIG. 20A shows IL-27 expression levels in human derived dendritic cells stimulated with TriDAP with and without LPS, while FIG. 20B shows IL-10 expression levels in the same cell population.

### DETAILED DESCRIPTION OF THE INVENTION

The inventors have surprisingly identified a reduction in the severity, and delay in onset, of the induced autoimmune disease, EAE, following administration of the NOD1 agonist, Tri-DAP. The reduction in the severity of EAE was correlated with a reduction in the Th1 and Th17 cell mediated responses. EAE is an autoimmune disease of the CNS and an animal model for MS. As such, the results obtained from the experimentation in the EAE animal model can be extrapolated to the treatment of multiple sclerosis in human subjects. The inventors have identified that Tri-DAP containing compounds have utility in the amelioration of conditions, such as autoimmune and chronic inflammatory diseases such multiple sclerosis, which are mediated by aberrant Th1 and Th17 T-cell responses, either due to pathology caused by the T cell subsets themselves, or by the immune response which is driven by the cytokines which cause the differentiation and activation of such T cells.

Without wishing to be bound by theory, the inventors predict that the down-regulation of Th1 and Th17-mediated pro-inflammatory immune responses which are causative of EAE in mice and autoimmune conditions such as EAE in humans, which results following the administration of Tri-DAP at the time of, and/or after autoimmune disease onset, is mediated by the modulation of the activity of antigen presenting cells, in particular dendritic cells (DCs), in inducing a T-cell mediated immune response. In particular, the administration of TriDAP causes the function of antigen presenting cells to be modified by virtue of reduced MHC class II expression on the surface of the antigen presenting cell, and therefore reduced presentation of antigen to T cells. Furthermore, the expression of the cytokine IL-27 is increased. The co-administration of at least one Toll-like Receptor agonist alongwith TriDAP (that is sequentially or subsequently to the administration of TriDAP) further enhances the expression of IL-27 by dendritic cells in a synergistic manner. The modulation of antigen presenting cell behaviour following exposure to TriDAP therefore results in a reduction in the ability of the antigen presenting cell to activate memory T cells, or cause the differentiation of naïve CD4+ T cells into T cells having the Th1 or Th17 phenotype.

The inventors have surprising identified that signalling mediated through NOD-1, following binding to NOD-1 of a NOD-1 agonist, such as Tri-DAP, or a related diaminopimelic acid (DAP)-containing muropeptide compound, results in the production of a cytokine profile which mediates an anti-inflammatory immune response. This observation by the inventors was entirely unexpected, as it is the accepted dogma in the field that the stimulation of NOD-1 by a NOD-1 agonist would result in the expression of a cytokine profile by the NOD-1 expressing cell which would mediate a pro-inflammatory immune response.

Further, the inventors have identified that the interaction of Tri-DAP with antigen presenting cells, such as dendritic cells in the presence of at least one TLR agonist enhances the production of the inhibitory (that is, anti-inflammatory) cytokines IL-10 and IL-27. The inventors further predict that enhanced IL-10 and/or IL-27 production by dendritic cells following TriDAP administration results in suppressed Th1 and Th17-mediated T-cell responses, and reduced Th1 and Th17 cellular infiltration to the CNS following treatment. Disease progression and pathology is therefore reduced.

This observed activity of Tri-DAP on APC has been identified by the inventors as having utility in the treatment and/or prophylaxis of autoimmune and chronic inflammatory diseases, such as, but not limited to, multiple sclerosis, rheumatoid arthritis, inflammatory bowel disease including Crohn's disease and/or ulcerative colitis and type 1 diabetes. The expression of both IL-10 and IL-27 are known to be associated with the onset of type 1 diabetes (Bettini and Vignali. Current Opinion in Immunology. 2009. 21. 612-618). Rheumatoid arthritis is a chronic autoimmune disease in which pro-inflammatory cytokines, such as TNF-a, IL-6 and IL-1 play dominant pathological roles. More recently, IL-17 has been suggested to play an important additional role in the induction and maintenance of RA. IL-17 is produced predominantly by T helper cells (Th17 cells). TNF-α has been shown in vitro to drive the production of IL-17 by equipping DC with the ability to differentiate T cells towards a Th17 phenotype. Surprisingly, the inventors have shown that TriDAP reduces the ability of dendritic cells to present antigen to T cells. Furthermore, TriDAP induces production of IL-27 which prevents the differentiation of naïve T cells towards the Th17 phenotype. Accordingly, in stimulating the production of cytokines such as IL-10 and IL-27, TriDAP has been shown to be an important negative regulator of IL-17 and IFN-γ production by T cells and the inventors therefore propose that this forms part of a negative feedback loop that limits the intensity and/or duration of Th17 and Th1 responses, and therefore provides a novel therapeutic approach for the treatment of rheumatoid arthritis.

L-ala-γ-D-Glu-mDAP (Tri-DAP) is a diaminopimelic-containing tripeptide present in the peptidoglycan of Gram-negative bacteria that is specifically recognised by NOD1 (NOD-1). As mentioned previously, NOD1 is a PRR located in the cytoplasm of APC, such as DCs, that functions primarily in initiating immune responses to microbial pathogen associated molecular patterns (PAMPs) upon ligand binding. The type of immune responses initiated by APC in response to PRR ligand binding plays a key role in the determination of the subsequent type of Ag-specific T-cell populations recruited. NOD1 ligand binding is usually associated with the activation of members of the MAPK family and the transcription factor NF-κB.

The inventors have made the surprising observation that administration of a composition comprising Tri-DAP results in a reduction of disease severity and progression of autoimmune disease, and in particular autoimmune disease conditions which are mediated by autoreactive T cells, as exemplified by an EAE disease model in mice. Accordingly, in certain embodiments, the present invention extends to the administration of a therapeutically effective amount of a composition containing Tri-DAP, or synthetic or non-synthetic analogues thereof, for the treatment and/or prophylaxis of autoimmune and chronic inflammatory disease in subjects requiring such treatment. In further embodiments, the reduction of clinical disease score and disease severity results from the inhibition or downregulation of Th1 and Th17-mediated immune responses is seen in mice presenting with the EAE disease model.

IL-27 is an inhibitory cytokine composed of p28 and EBI3 that can potently suppress the effector phase of EAE in vivo and, thus, has been identified as having therapeutic potential in relation to autoimmune diseases such as MS (Fitzgerald et al., 2007). IL-27 is produced by antigen-presenting cells and signals through its receptor, IL-27R, expressed by monocytes/macrophages, dendritic cells, T and B cells, natural killer cells, mast cells, and endothelial cells. IL-27 has been shown to play a suppressive role in EAE as demonstrated by more severe disease in IL-27R-deficient mice. Exogenous IL-27 potently suppressed the ability of encephalitogenic lymph node spleen cells to transfer EAE. IL-27 significantly inhibited IL-23-driven IL-17 production by myelin-reactive T cells thereby suppressing their encephalitogenicity in adoptive transfer EAE. Furthermore, it has been demonstrated that a strong suppressive effect of IL-27 on active EAE in vivo when delivered by sub-cutaneous osmotic pump. IL-27-treated mice had reduced CNS inflammatory infiltration and, notably, a lower proportion of Th17 cells.

In the present invention the inventors have surprisingly observed that administration of a compound containing Tri-DAP in conjunction with a TLR agonist in vitro, or a suitable adjuvant in vivo, results in enhanced production of IL-27 by BMDC. Accordingly, in certain embodiments of the present invention, the composition containing Tri-DAP may be administered in conjunction with a pharmaceutically acceptable TLR agonist.

In certain embodiments, the Toll-like Receptor (TLR) agonist is specific for TLR2, TLR4 or TLR9. The TLR2 receptor is a heterodimer found in combination with Toll-like Receptor 1 or Toll-like Receptor 6. Bacterial lipopeptides are the main agonists for TLR2 containing receptors. Examples of TLR2 agonists include, but are not limited to: mycoplasma macrophage-activating lipoprotein-2, highly purified soluble peptidoglycan, lipoteichoic acid, outer surface protein A from Borrelia burgdorferi, tripalmitoyl-cysteinyl-seryl-(lysyl)3-lysine (Pam3CSK4, P3CSK4), dipalmitoyl-CSK4 (Pam2CSK4, P2-CSK4), and monopalmitoyl-CSK4 (PCSK4) as well as lipopolysaccharide and inactivated bacteria.

The classic agonist for TLR4 is bacterial lipopolysaccharide (LPS), which refers to a family of substances containing lipid A and the like. An exemplary form of LPS is E. coil B:O111 (Sigma Chemicals). A less toxic TLR4 agonist is a monophosphoryl lipid A (MPL) compound. The synthetic adjuvant, ASO2 (GlaxoSmithKline, United Kingdom), contains MPL as a component.

Immunostimulatory oligonucleotides or polynucleotides such as CpG-containing oligodeoxynucleotides (CpG ODN) are the typical agonists for TLR9. More generally, they are called immunostimulatory sequences of oligodeoxynucleotides (ISS-ODN) because many immunostimulatory oligonucleotides (ODNs) do not contain a CpG motif. Typically, the ODN is a synthetic thiophosphorylate-linked compound. However, many types of DNA and RNA can activate TLR9 including bacterial DNA, liposomal vertebrate DNA, insect DNA, chlamydia polynucleotides and others.

Typically the TLR agonist is a TLR2, TLR4 or a TLR9 agonist capable of inducing IL-27 production by dendritic cells. In certain embodiments the TLR agonist may be selected from any one or more of LPS (lipopolysaccharide), CpG motifs including CpG-containing oligodeoxynucleotides (CpG ODN), dsRNA, Poly (I:C) and Pam-3Cys.

In certain further embodiments the administration of the Tri-DAP containing compound may be accompanied with the administration of at least on ERK inhibitor. The ERK inhibitor may be provided simultaneously with or sequentially to the administration of the Tri-DAP containing compound. At least one TLR agonist, as described hereinbefore, may also be administered along with the Tri-DAP containing compound and ERK inhibitor.

Th1 cells confer protection against intracellular infection by activating the microbicidal activity of Macrophages and driving proliferation of CD8+ cytotoxic T-cells, but are also associated with inflammatory responses and autoimmune disorders. As a pro-inflammatory mediator, exaggerated Th1 cell responses can be detrimental to the host. Th1-polarising factors include IL-12, IL-18, type I interferons and cell-surface expressed intercellular adhesion molecule 1 (ICAM1). Th1 cells produce cytokines, in particular IFN-γ. Th17 cells are thought to be involved in early, tissue-specific inflammatory responses, resulting in the recruitment and activation of neutrophils. In addition, Th17 cells have recently been identified as particularly potent inducers of autoimmunity. Th17-polarising factors implicated to date include IL-23, IL-6, IL-1, IL-21 and TGF-β. Th17 cells produce cytokines, in particular IL-17, but also IL-22 and TNF-α.

The inventors of the present invention have surprisingly observed that administration of a compound containing Tri-DAP to animals suffering from autoimmune disease results in an inhibition or down-regulation of production of the Th1 and Th17 associated cytokines IFN-γ and TNF-α, and IL-17, respectively. Accordingly, in certain embodiments the suppression of the Th1 and Th17-mediated immune responses following Tri-DAP treatment in the present invention results in the inhibition or downregulation of at least one cytokine from the group comprising IFNγ, TNFα and IL-17. In certain further embodiments, the suppression of the Th1 and Th17-mediated immune responses following Tri-DAP treatment in the present invention results in a reduction in the numbers of IFNγ and IL-17 producing T-cells infiltrating the CNS during autoimmune and chronic inflammatory disease.

### Peptidomimetics and Synthetic Analogues

Peptide analogues, such as peptidomimetics or peptide mimetics are non-peptide compounds with properties representative of a template peptide. Such peptide analogues are typically developed using computerised molecular modelling. Peptidomimetics which are structurally similar to peptides which have affinity and binding specificity to NOD1, such as TriDAP, may be used to mediate similar prophylactic and therapeutic effects to polypeptides and proteins which are determined to have such Th1 and Th17 inhibitory function.

Peptidomimetics are typically structurally similar to a template peptide, but have one or more peptide linkages replaced by an alternative linkage, by methods which are well known in the art. For example, a peptide which has binding specificity to a NOD1 epitope, such as TriDAP, may be modified such that it comprises amide bond replacement, incorporation of non peptide moieties, or backbone cyclisation. Suitably if cysteine is present, the thiol of this residue can be capped to prevent damage of the free sulphate group. A peptide may further be modified from the natural sequence to protect the peptides from protease attack.

Accordingly, a peptide compound used as a NOD1 agonist in the present invention may be further modified using at least one of C and/or N-terminal capping, and/or cysteine residue capping. Furthermore, a peptide for use in the present invention may be capped at the N terminal residue with an acetyl group. Suitably, a peptide of and for use in the present invention may be capped at the C terminal with an amide group. Suitably, the thiol groups of cysteines are capped with acetamido methyl groups.

### Analogues, Derivatives, Mimetics, and Variants of Tri-DAP

The present invention extends to analogues, derivatives, fragments, and variants of Tri-DAP containing compositions for use in the present invention. A derivative, fragment or variant of Tri-DAP as defined herein is understood to mean any compound, molecule or macromolecule consisting of a portion Tri-DAP which exhibits the observed immunosuppressive properties of Tri-DAP. Such derivatives, fragments or variants may be prepared by a person skilled in the art using any one of a number of techniques commonly known to the skilled person. Such fragments, variants, analogues or derivatives mediate an identical or substantially similar biological function to that of Tri-DAP containing compositions when acting on the cells of the innate immune system. As such, the present invention is further intended to encompass, in addition to the use of Tri-DAP and M-TriDAP containing compositions, the use of homologues and analogues of such compounds. In this context, homologues are molecules having substantial structural similarities to the above-described compounds and analogues are molecules having substantial biological similarities regardless of structural similarities.

In certain further embodiments, the compounds of the invention may be modulated by exchange or substitution of certain amino acid residues. As is well understood, homology at the amino acid level is generally in terms of amino acid similarity or identity. Similarity allows for ‘conservative variation’, such as substitution of one hydrophobic residue for another or the substitution of one polar residue for another.

Non-peptide mimetics are provided within the scope of the invention. In certain embodiments, the compounds of the invention can be modified by substituting an alanine (ala, A) residue for a serine (ser, S) residue or a valine (val, V) reside. In certain further embodiments, the glutamic acid (glu, E) residue may be replaced by an aspartate (Asp, D) residue.

### Combination Medicaments

As described hereinbefore, in certain aspects, the present invention extends to combinational therapies wherein compositions or methods of the invention extend to the administration of further compounds which modulate the activity of antigen presenting cells, particularly dendritic cells, wherein said further compounds are administered in combination with at least one TLR agonist and/or at least one inhibitor of ERK MAPK.

Typically the primary and secondary therapeutic compositions are given contemporaneously. In certain embodiments, the primary therapeutic composition (i.e. the compound which modulates the functional activity of APC) and the secondary therapeutic, which could be, for example, a TLR agonist, are administered simultaneously. In certain further embodiments, they are administered sequentially.

In certain embodiments, the combination therapy may comprise a composition containing Tri-DAP, or a synthetic or non-synthetic analogue thereof or a peptidomimetic, which is co-administered to a subject along with at least one of: a TLR agonist (such as an agonist specific for TLR2, TLR4 or TLR9), a cytokine inhibitor (such as, but not limited to an inhibitor of IL-1, TNF-α, IL-17, IFN-γ, IL-23, IL-6, TGF-β and IL-12), an ERK inhibitor, such as PD98059 or U0126, a growth factor inhibitor, an immunosuppressor, such as an antibody, an anti-inflammatory, an enzymatic inhibitor, a steroid, a non-steroid anti-inflammatory drug, a metabolic inhibitor, a cytotoxic agent or a cytostatic agent.

A person of relevant skill in the field will recognise that the administration to a subject of a combination therapy can be advantageous in that it permits administration of a lower dose of therapeutic to a subject in order to achieve and associated therapeutically effective effect. The administration of a lower combined dose also results in the subject being exposed to a lower toxicity level derived from the administered compound. Furthermore, as the secondary therapeutic compounds which are administered as part of the combination therapy provided by the invention target different pathways, there is likely to be a synergistic improvement in the overall efficacy of the therapy. An improvement in efficacy would again result in the need for a lower dose to be administered and as such an associated reduction in toxicity.

### Pharmaceutical Compositions

The present invention extends to a pharmaceutical composition comprising a compound which inhibits or down-regulates Th1 and Th17 cell mediated responses in autoimmune or chronic inflammatory disease through modulation of APC activity. Pharmaceutical compositions according to and for use in accordance with the present invention may comprise, in addition to active ingredient (i.e. an inhibitor of Th1 and Th17 cell), a pharmaceutically acceptable excipient, carrier, buffer stabiliser or other materials well known to those skilled in the art. Examples of suitable pharmaceutical carriers include; water, glycerol, ethanol and the like.

The composition of the present invention may be administered to a patient in need of treatment via any suitable route. As detailed herein, it is preferred that the composition is administered parenterally. Examples of preferred routes for parenteral administration include, but are not limited to; intravenous, intracardial, intraarterial, intraperitoneal, intramuscular, intracavity, subcutaneous, transmucosal, inhalation or transdermal.

Routes of administration may further include topical and enteral, for example, mucosal (including pulmonary), oral, nasal, rectal. The formulation may be a liquid, for example, a physiologic salt solution containing non-phosphate buffer at pH 6.8-7.6, or a lyophilised or freeze dried powder.

In certain embodiments, the composition is deliverable as an injectable composition. For intravenous injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection or, Lactated Ringer's injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatine or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

Examples of the techniques and protocols mentioned above and other techniques and protocols which may be used in accordance with the invention can be found in Remington's Pharmaceutical Sciences, 18th edition, Gennaro, A. R., Lippincott Williams & Wilkins; 20th edition ISBN 0-912734-04-3 and Pharmaceutical Dosage Forms and Drug Delivery Systems; Ansel, H. C. et al. 7th Edition ISBN 0-683305-72-7, the entire disclosures of which is herein incorporated by reference.

The composition may also be administered via microspheres, liposomes, other microparticulate delivery systems or sustained release formulations placed in certain tissues including blood.

Dosage regimens can include a single administration of the composition of the invention, or multiple administrative doses of the composition. The compositions can further be administered sequentially or separately with other therapeutics and medicaments which are used for the treatment of the condition for which the composition of the present invention is being administered to treat.

The actual amount administered, and rate and time-course of administration, will depend on the nature and severity of what is being treated. Prescription of treatment, e.g. decisions on dosage etc, is ultimately within the responsibility and at the discretion of general practitioners and other medical doctors, and typically takes account of the disorder to be treated, the condition of the individual patient, the site of delivery, the method of administration and other factors known to practitioners.

### DEFINITIONS

Unless otherwise defined, all technical and scientific terms used herein have the meaning commonly understood by a person who is skilled in the art in the field of the present invention.

Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers.

As used herein, terms such as “a”, “an” and “the” include singular and plural referents unless the context clearly demands otherwise. Thus, for example, reference to “an active agent” or “a pharmacologically active agent” includes a single active agent as well as two or more different active agents in combination, while references to “a carrier” includes mixtures of two or more carriers as well as a single carrier, and the like.

The term “antigen” as used herein is any organic or inorganic molecule capable of stimulating the immune response. The term “antigen” as used herein extends to any peptide, polypeptide, protein, nucleic acid molecule, carbohydrate molecule, organic or inorganic molecule capable of stimulating an immune response.

As used herein, the term “therapeutically effective amount” means the amount of an agent, binding compound, small molecule, fusion protein or peptidomimetic of the invention which is required to suppress a Th1 and/or Th17-mediated inflammatory condition or autoimmune disease.

As used herein, the term “prophylactically effective amount” relates to the amount of a composition which is required to prevent the initial onset, progression or recurrence of a Th1 and/or Th17-mediated inflammatory condition, for example an autoimmune disease, such as multiple sclerosis.

A “Th1 and/or Th17-mediated inflammatory condition” as defined herein means a chronic inflammatory condition or autoimmune disease which is mediated in totality or in part by autoreactive Th1 and/or Th17 T cells which are specific for a self antigen. Examples of Th1 and/or Th17-mediated inflammatory conditions are provided hereinbefore and include, but are not limited to multiple sclerosis, rheumatoid arthritis, inflammatory bowel disease, type 1 diabetes and psoriasis.

As used herein, the term “treatment” and associated terms such as “treat” and “treating” means the reduction of the progression, severity and/or duration of a Th1 and/or Th17 mediated autoimmune disease or chronic inflammatory condition or at least one symptom thereof, wherein said reduction or amelioration results from the administration of a compound which induces the production of the inhibitory cytokine, IL-27, in particular by an antigen presenting cell (APC), particularly a dendritic cell (DC) or a macrophage. The IL-27 production may prevent the maturation of naïve T cells into the Th1 or Th17 phenotype, or may prevent the activation of Th1 or Th17 T cells.

The term ‘treatment’ therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition.

As used herein, the term “immune cell” includes cells that are of haematopoietic origin and that play a role in the immune response. Immune cells include lymphocytes, such as B cells and T cells; natural killer cells; myeloid cells, such as monocytes, macrophages, dendritic cells, eosinophils, mast cells, basophils, and granulocytes.

As used herein, the term “T cell” extends to CD4+ T cells, CD8+ T cells, γδ (gamma delta) T cells and NK (natural killer) T cells. The term T cell also includes both T helper 1 type T cells and T helper 2 type T cells and also Th-IL17 cells.

As used herein, the term “antigen-presenting cell” or “antigen-presenting cells” or its abbreviation “APC” or “APCs” refers to a cell or cells capable of endocytotic adsorption, processing and presenting of an antigen. The term includes professional antigen presenting cells for example; B lymphocytes, monocytes, dendritic cells (DCs) and Langerhans cells, as well as other antigen presenting cells such as keratinocytes, endothelial cells, glial cells, fibroblasts and oligodendrocytes. The term “antigen presenting” means the display of antigen as peptide fragments bound to MHC molecules, on the cell surface. Many different kinds of cells may function as APCs including, for example, macrophages, B cells, follicular dendritic cells and dendritic cells.

As used herein, the term “immune response” includes T cell mediated and/or B cell mediated immune responses that are influenced by modulation of T cell co-stimulation. The term immune response further includes immune responses that are indirectly effected by T cell activation such as antibody production (humoral responses) and the activation of cytokine responsive cells such as macrophages.

As used herein, the term “dendritic cell” or “dendritic cells” (DC) refers to a dendritic cell or cells in its broadest context and includes any DC that is capable of antigen presentation. The term includes all DC that initiate an immune response and/or present an antigen to T lymphocytes and/or provide T-cells with any other activation signal required for stimulation of an immune response. Reference herein to “DC” should be read as including reference to cells exhibiting dendritic cell morphology, phenotype or functional activity and to mutants or variants thereof. The morphological features of dendritic cells may include, but are not limited to, long cytoplasmic processes or large cells with multiple fine dendrites. Phenotypic characteristics may include, but are not limited to, expression of one or more of MHC class I molecules, MHC class II molecules, CD11c, B220, CD8-alpha, CD1, CD4.

As herein defined, the phrase “upregulates cytokine production” or a variant thereof, means that the level of production of a cytokine is increased when compared to a previous known value. Further, as herein defined, the phrase “downregulates cytokine production” means that an agent causes a reduction in the production of a cytokine when compared to a previously known value.

A “subject” in the context of the present invention includes and encompasses mammals such as humans, primates and livestock animals (e.g. sheep, pigs, cattle, horses, donkeys); laboratory test animals such as mice, rabbits, rats and guinea pigs; and companion animals such as dogs and cats. It is preferred for the purposes of the present invention that the mammal is a human. The term “subject” is interchangeable with the term “patient” as used herein.

### EXAMPLES

The present invention will now be described with reference to the following examples which are provided for the purpose of illustration and are not intended to be construed as being limiting on the present invention.

### Example 1

### Treatment with the NOD1 Agonist Tri-DAP Attenuates Experimental Autoimmune Encephalomyelitis (EAE)

This experiment was designed to identify whether Tri-DAP treatment could reduce the clinical score of disease severity following induction of EAE in mice.

### Material and Methods:

EAE was induced in C57BL/6 mice by subcutaneous (s.c) injection with 150 μg of MOG₃₅₋₅₅ in CFA supplemented with 4 mg/ml H37 Ra M. tuberculosis on day 0. All mice were injected intra-peritoneally (i.p) with Pertussis toxin (PT) on days 0 and 2. One group of mice were untreated and the second group were given Tri-DAP (100 μg/mouse) on day 0 in MOG/CFA emulsion and again on days 1, 2 and every 2 days thereafter. Clinical scores were assessed daily and body weights were recorded. Disease severity was graded as follows: grade 0: normal, grade 1: limp tail, grade 2: wobbly gait; grade 3: hind limb weakness; grade 4: hind limb paralysis; grade 5: tetraparalysis/death. † indicates control mice were sacrificed due to severity of EAE disease.

### Results:

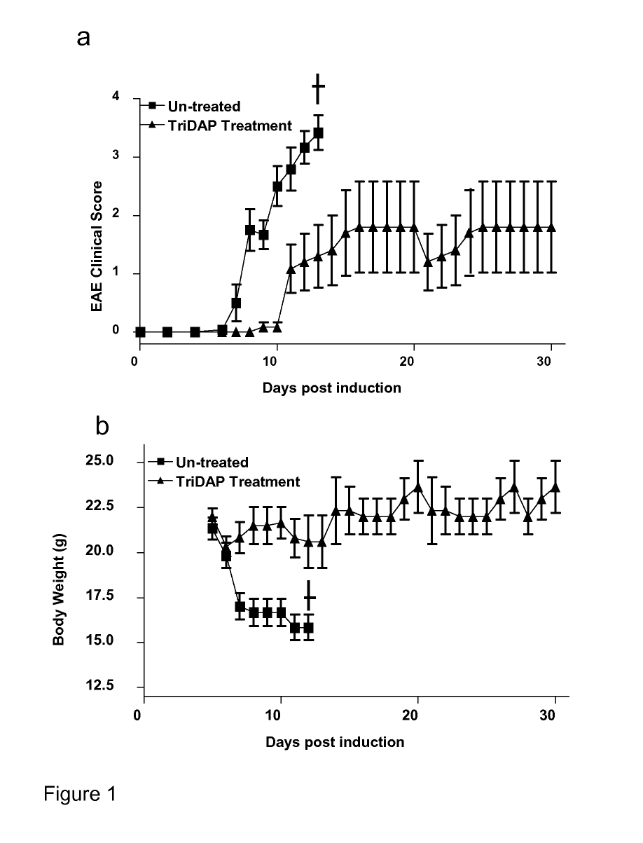

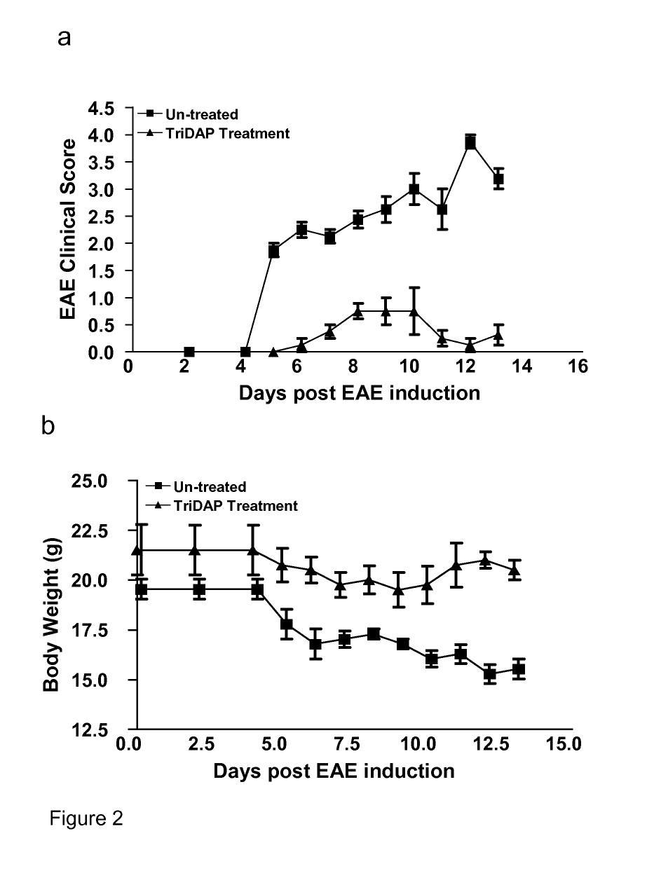

Untreated mice developed clinical signs of EAE after 5-6 days and the severity of the disease rapidly peaked to a clinical score of 3.5-4 by day 12 (FIGS. 1 and 2). In contrast, Tri-DAP treated mice did not show any clinical signs of disease until 11 days post induction (FIG. 1) and the severity of disease was lower than that observed in the untreated control mice (FIGS. 1 and 2). Exacerbated EAE in control mice was also evident by the dramatic weight loss in the control mice when compared to the Tri-DAP treated mice (FIGS. 1 and 2).

These results demonstrate that Tri-DAP treatment at and following EAE induction attenuates the development of EAE disease.

### Example 2

### Treatment with the NOD1 Agonist Tri-DAP Suppresses MOG-Specific Inflammatory Cytokines Following EAE Induction

This experiment was designed to determine the effect of Tri-DAP treatment on the production of Th1 and Th17 specific inflammatory cytokines following EAE induction.

### Materials and Methods:

EAE was induced in C57BL/6 mice by s.c injection with 150 μg of MOG₃₅₋₅₅ in CFA supplemented with 4 mg/ml H37 Ra M. tuberculosis on day 0. All mice were injected i.p with PT on days 0 and 2. One group of mice were untreated, the second group were given 100 μg Tri-DAP 6 hours before immunization and a third group were given Tri-DAP (100 μg/mouse) on day 0 in MOG/CFA emulsion and again on days 1, 2 and 4. Mice were sacrificed on day 5 and lymph nodes cells were stimulated with MOG peptide (Myelin oligodendrocyte glycoprotein) (20-100 μg/ml) or as negative and positive controls, medium only or PMA and anti-CD3 respectively. After 72 hours supernatants were removed and analyzed for IL-17, TNF-α (TNF-alpha) and IFN-γ (interferon gamma) by ELISA.

### Results:

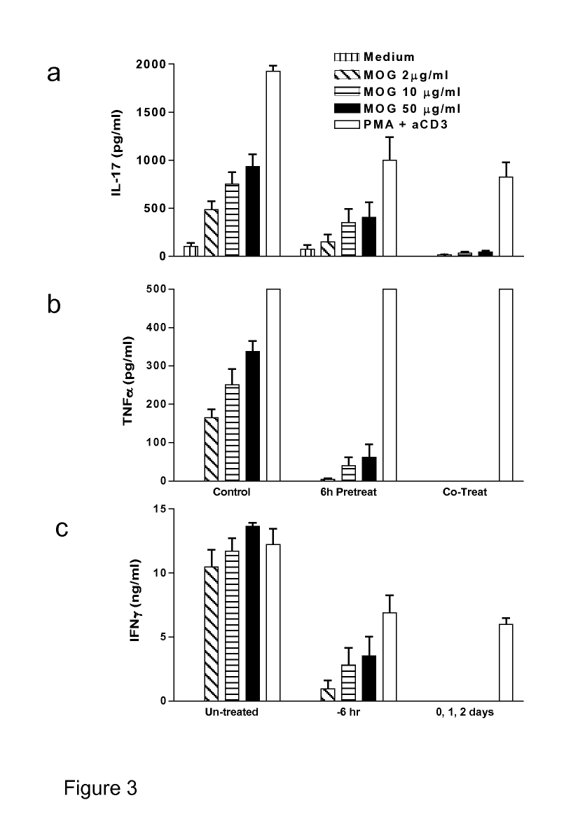

A robust MOG-specific IL-17, IFN-γ and TNF-α response was detected in the inguinal lymph nodes of control EAE mice 5 days post EAE induction (FIG. 3). In contrast, mice that were treated with Tri-DAP 6 hours prior to EAE induction had significantly reduced antigen-specific T cell responses when compared to control EAE mice. Tri-DAP treatment on days 0, 1, 2 and 4 had no detectable MOG-specific T cell responses (FIG. 3).

These results demonstrate that treatment with Tri-DAP suppresses the production of the pro-inflammatory cytokines IL-17, IFN-γ and TNF-α in the lymph nodes of mice following EAE induction.

### Example 3

### Treatment with the NOD1 Agonist Tri-DAP Suppresses IL-17-expressing and IFNγ-Expressing T Cells in the Brains of Mice with EAE

This experiment was designed to determine the effect of Tri-DAP treatment on various IFN-γ and IL-17 producing T-cell populations in the brains of mice following EAE induction.

### Materials and Methods:

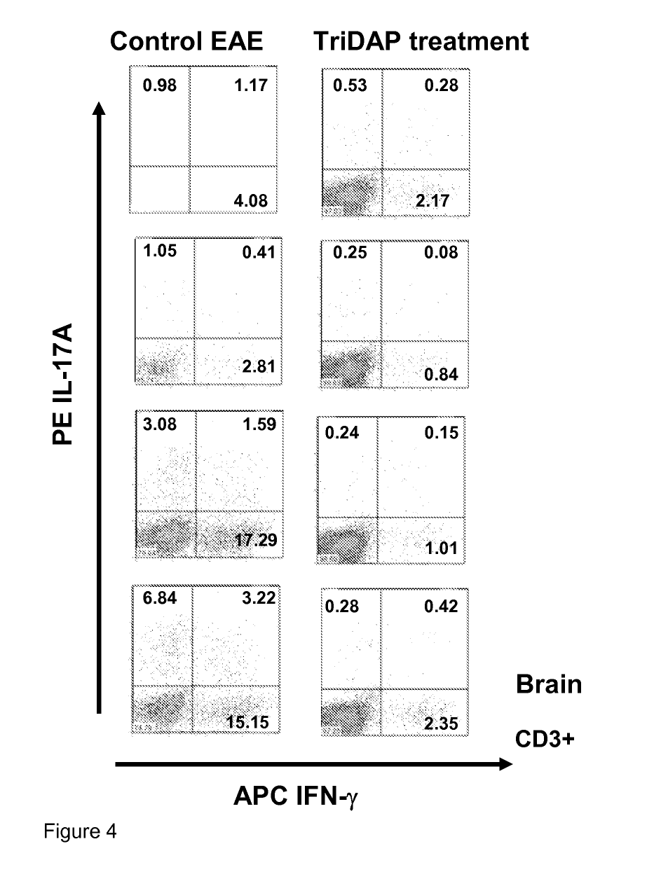

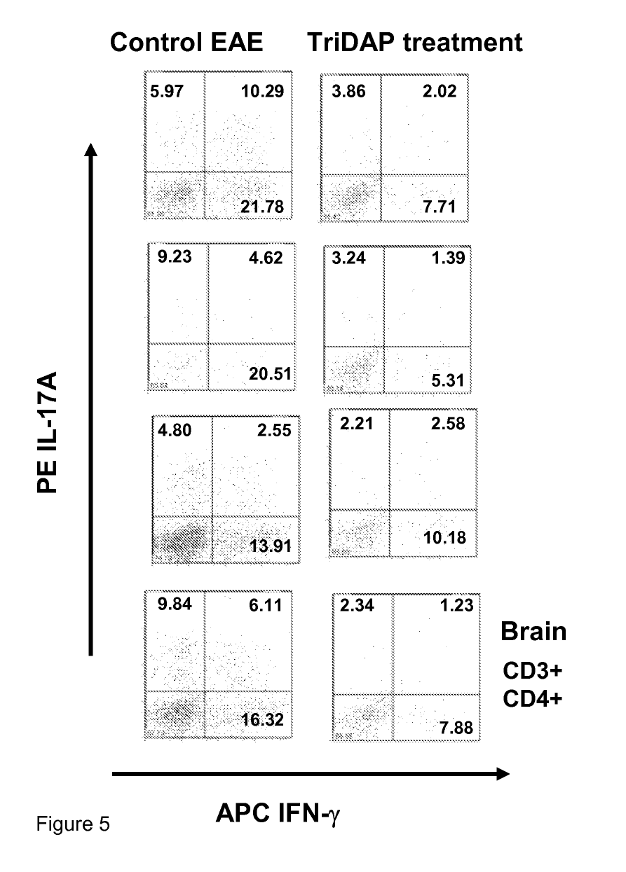

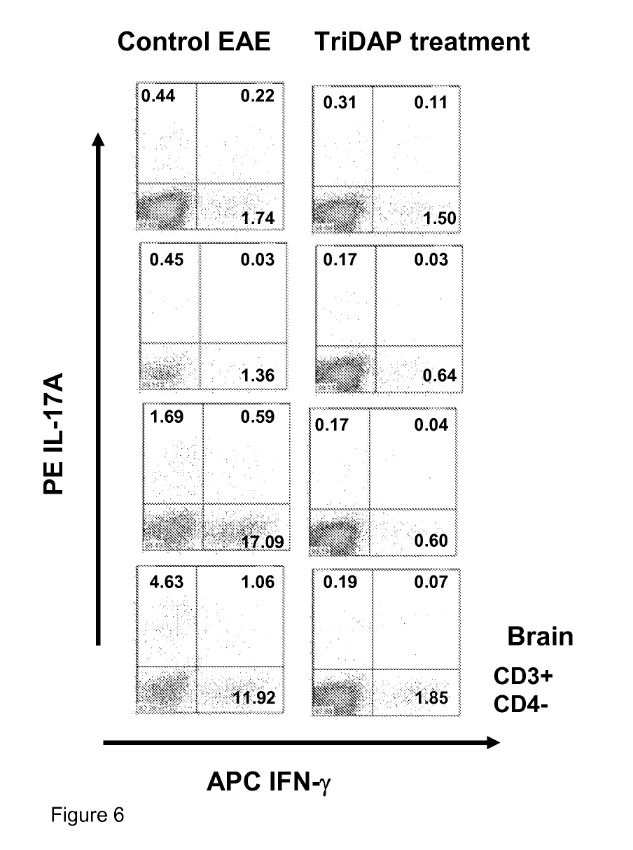

Mononuclear cells were isolated from the brains of control EAE or Tri-DAP treated EAE mice 12 days post EAE induction. Cells were restimulated overnight with PMA/ionomycin and incubated with Brefeldin A. Cells were stained with anti-CD3 (FIG. 4), or anti-CD3 and anti-CD4 (FIGS. 5 and 6). Cells were then fixed and permeabilized, stained intracellularly with anti-IFN-γ and anti-IL-17 and analysed by flow cytometry.

### Results:

In all T-cell populations examined (CD3⁺ cells in FIG. 4, CD3⁺ CD4⁺ cells in FIG. 5 and CD3⁺ CD4⁻ cells in FIG. 6) Tri-DAP treatment suppressed IFN-γ and IL-17 production (FIGS. 4-6).

These results demonstrate that treatment with Tri-DAP suppresses the infiltration of IFN-γ and IL-17-producing T-cell populations in the CNS of mice following EAE induction.

### Example 4

### Treatment with the NOD1 Agonist Tri-DAP enhances EBI3 mRNA Expression in the Inguinal Lymph Nodes of Mice 72 Hours Post EAE Induction

This experiment was designed to assess the effect of Tri-DAP treatment on EBI3 gene expression in the inguinal lymph nodes of mice following EAE induction.

### Materials and Methods:

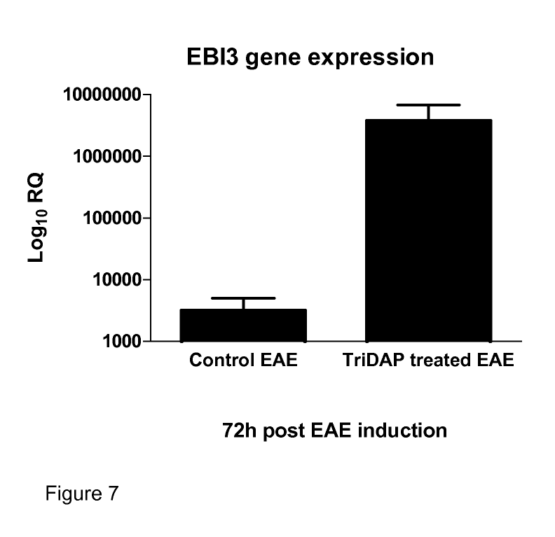

EAE was induced in C57BL/6 mice by subcutaneous injection with 150 μg of MOG35-55 in CFA supplemented with 4 mg/ml H37 Ra M. tuberculosis on day 0. All mice were injected i.p with PT on days 0 and 2. One group of mice were untreated and the second group were given Tri-DAP (100 μg/mouse) given on day 0 in MOG/CFA emulsion and again on days 1 and 2. Three mice from each group were sacrificed 72 hours post EAE induction. The inguinal lymph nodes were removed and collected in Trizol. EBI3 mRNA was evaluated by real time PCR normalized to 18S rRNA. Data are shown as fold induction over naïve C57BL/6 inguinal lymph nodes (FIG. 7).

### Results:

When EBI3 gene expression was examined in the inguinal lymph nodes of control and Tri-DAP treated mice there was a strong fold increase in EBI3 gene expression in the lymph nodes of Tri-DAP treated mice when compared to control mice (FIG. 7).

These results provide evidence that the ability of Tri-DAP to attenuate EAE may be dependent on the production of the inhibitory cytokine IL-27.

### Example 5

### Treatment with the NOD1 Agonist Tri-DAP Enhances LPS Induced EBI3 and IL-27p28 Expression in Dendritic Cells

This experiment was designed to assess the effect of Tri-DAP treatment on EBI3 and IL-27p28 gene expression in BMDC stimulated with Toll-like Receptor (TLR) agonists.

### Materials and Methods:

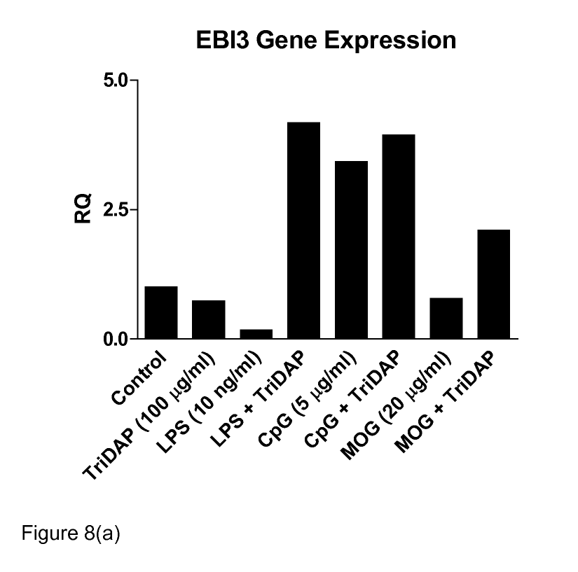

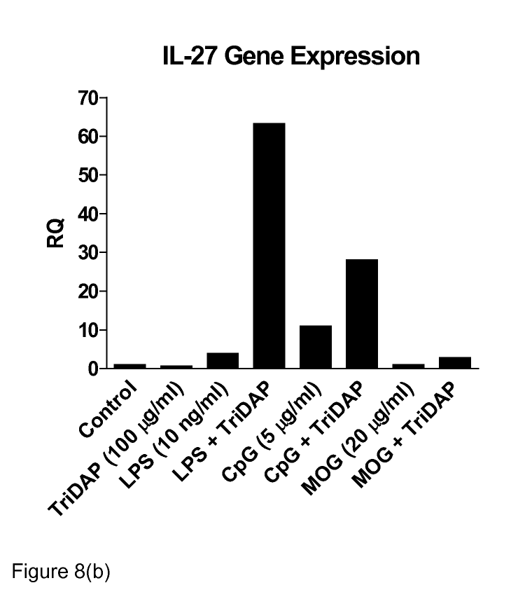

Bone marrow derived DC (1×10⁶ cell/ml) were pretreated with Tri-DAP (100 μg/ml) for 24 hours prior to stimulation with the TLR agonists LPS (10 ng/ml) and CpG (5 μg/ml), and the MOG35-55 peptide (20 μg/ml) or medium alone (control) for 6 hours. EBI3 (FIG. 8 (a)) and IL-27p28 (FIG. 8 (b)) mRNA were evaluated by real time PCR normalized to 18S rRNA. Data are shown as fold induction over DC cultured in medium only.

### Results:

An enhancement of LPS induced EBI3 (FIG. 8 (a)) and IL-27p28 (FIG. 8 (b)) gene expression was observed in BMDC following Tri-DAP treatment. These results provide further evidence that Tri-DAP treatment of DCs induces production of the pro-inflammatory inhibitory cytokine IL-27.

### Example 6

### Inhibition of ERK Enhances Tri-DAP Synergy with LPS for Induction of EBI3 and IL-27p28 Gene Expression in DC

This experiment was designed to assess the role of ERK activation in the enhancement of LPS induced EBI3 and IL-27p28 gene expression in BMDC by Tri-DAP.

### Materials and Methods:

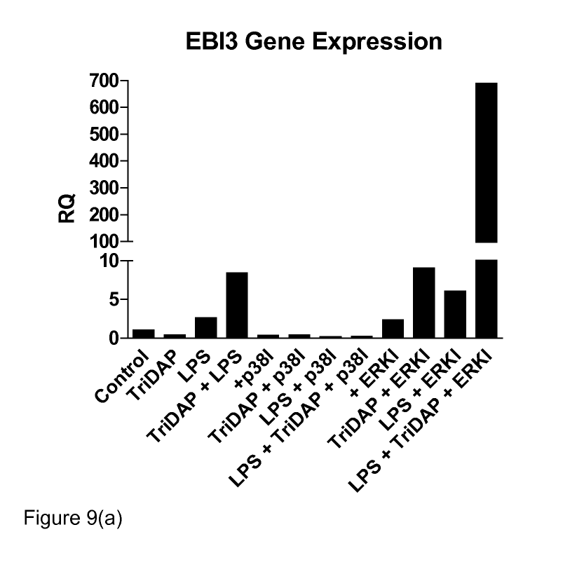

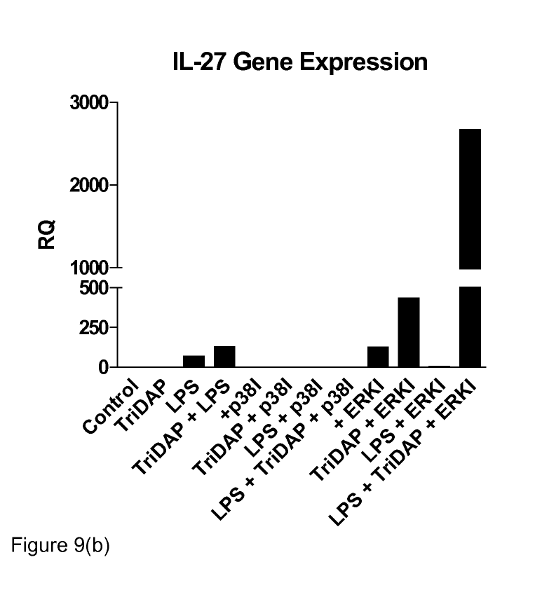

BMDC (1×10⁶) were pretreated with either the MAPK inhibitor, SB203580 (5 mM) or the MEK1/2 inhibitor, U0126 (5 mM) for 30 minutes prior to stimulation with TriDAP (100 mg/ml) or medium alone. After 24 hours the cells were then stimulated with LPS (lipopolysaccharide) (100 ng/ml), CpG (5 mg/ml) or medium alone. EBI3 and IL-27p28 mRNA were evaluated by real time PCR normalized to 18S rRNA. Data are shown as fold induction over DC cultured in medium only (FIG. 9 (a) and FIG. 9 (b)).

### Results:

Inhibition of ERK greatly enhances Tri-DAP synergy with LPS for both EBI3 and IL-27p28 gene expression in DC (FIG. 9 (a) and FIG. 9 (b)). Furthermore, the data showed that addition of an ERK inhibitor significantly enhances Tri-DAP induced or Tri-DAP and TLR agonist induced IL-27 production by the dendritic cells (DC).

### Example 7

### Enhancement of TLR Agonist Induced Cytokine Production in BMDC by Tri-DAP is Dependent Upon p38 and Partially Dependent Upon ERK

This experiment was designed to assess the role of the MAPKs p38 and ERK in the Tri-DAP-mediated enhancement of LPS induced IL-12p40, IL-12p70, IL-23 and IL-10 production by BMDC.

### Materials and Methods:

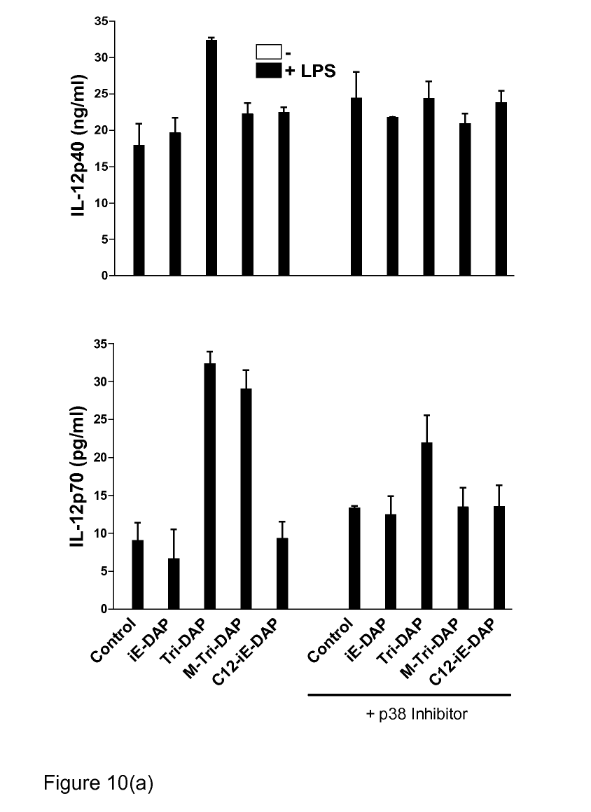

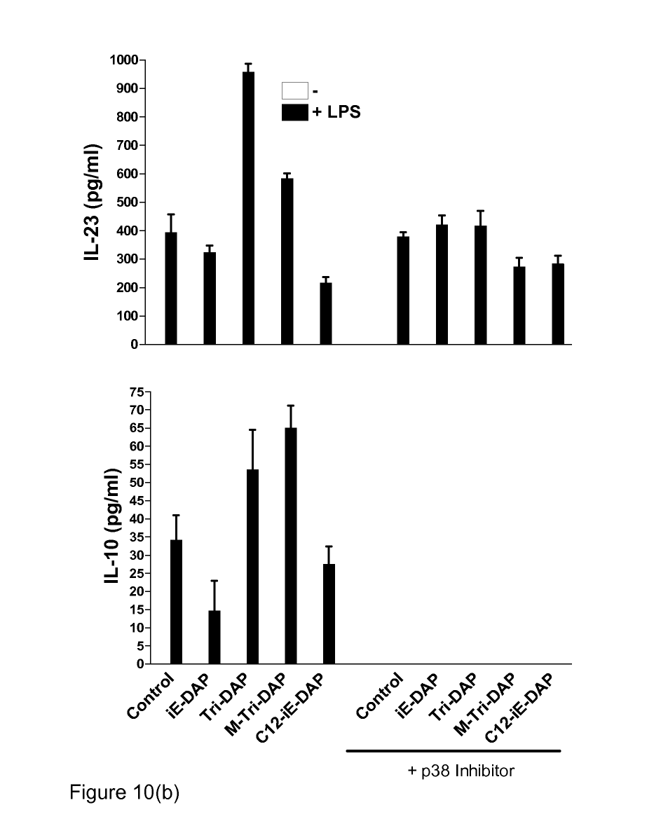

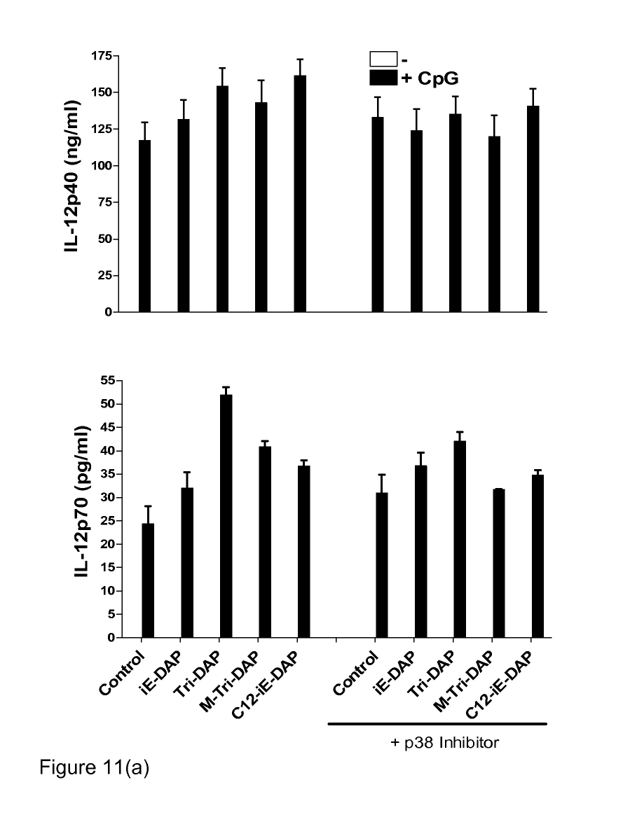

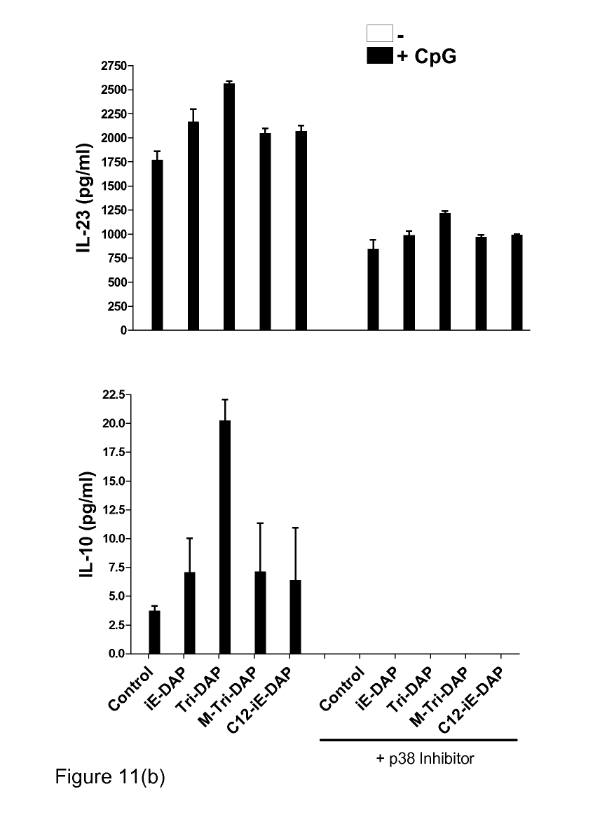

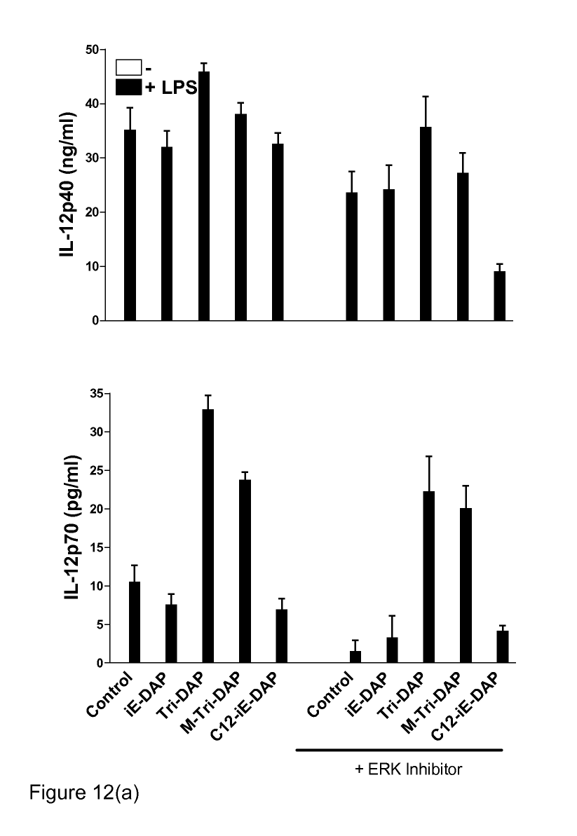

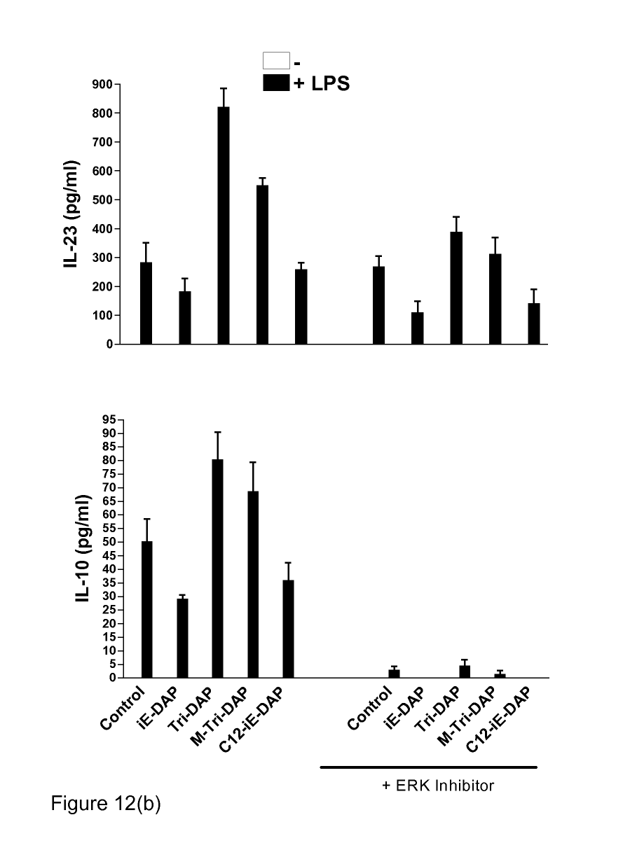

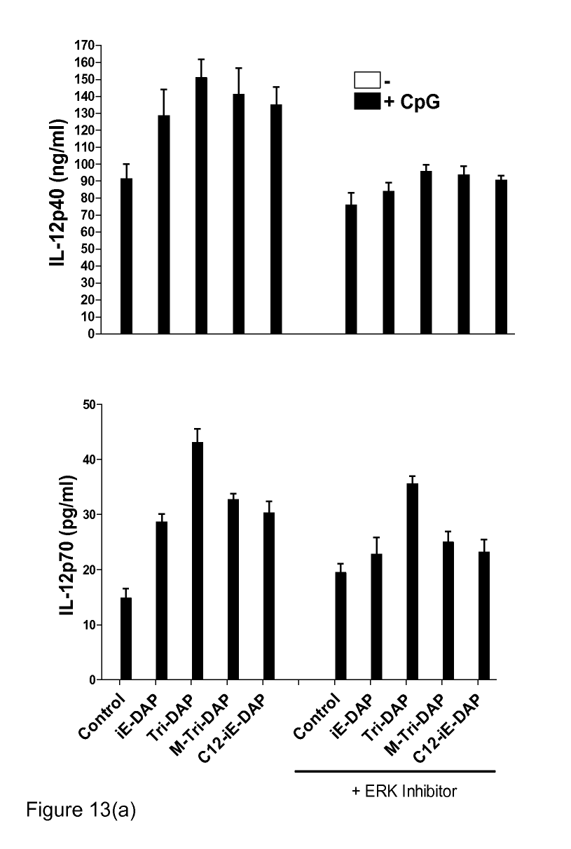

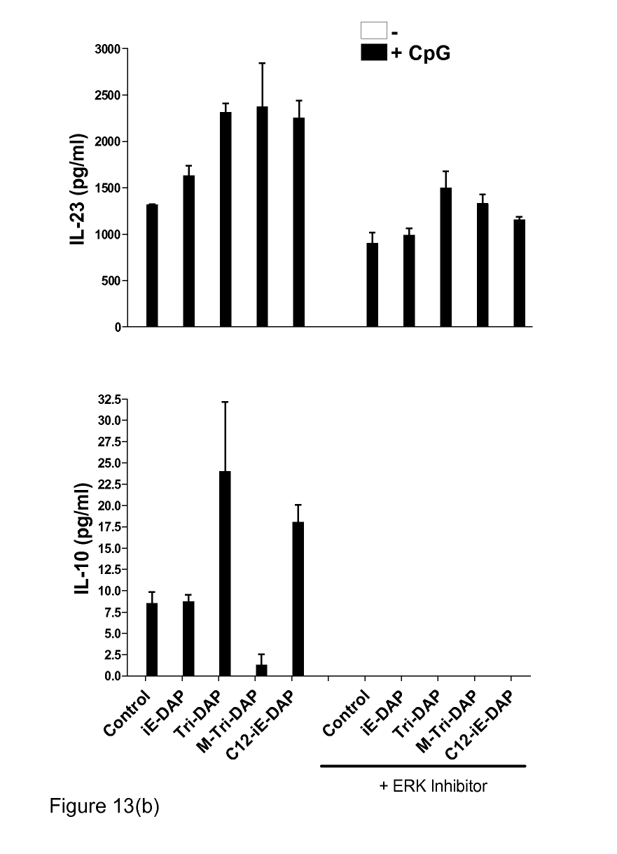

BMDC (1×10⁶) were pretreated with the p38 inhibitor, SB203580 (5 μM—FIGS. 10-11), or the ERK inhibitor UO126 (5 μM—FIGS. 12-13) for 30 minutes prior to stimulation with Tri-DAP (100 μg/ml) or medium alone. After 2 hours the cells were then stimulated with LPS (100 ng/ml—FIGS. 10 and 12), with the TLR agonist CpG (5 μg/ml—FIGS. 11 and 13) or with medium alone. Supernatants were collected after 24 hours and IL-12p40, IL-23, IL-12p70 and IL-10 concentrations were quantified by ELISA.

### Results:

Pretreatment of BMDC with Tri-DAP enhances both LPS and CpG production of the IL-12 family members, IL-12p40, IL-12p70 and IL-23 (FIGS. 10-13). The observed synergy for Tri-DAP with either LPS or CpG is partially dependent on p38 (FIGS. 10-11). Enhancement of LPS induced IL-23 production by Tri-DAP is suppressed by the addition of the ERK inhibitor, U0126 (FIG. 12 (b)). In contrast, enhancement of LPS induced IL-12p70 is only marginally affected in the presence of the ERK inhibitor (FIG. 12(a)). Both LPS and CpG induced IL-10 production by DC is enhanced by Tri-DAP and this enhancement is completely abolished when p38 is inhibited (FIGS. 10-11). A similar role for ERK is evident in the synergy observed for IL-10 production when DC are co-stimulated with Tri-DAP and LPS or CpG. IL-10 production by Tri-DAP with LPS or with CpG is suppressed in the presence of the ERK inhibitor (FIGS. 12-13).

These results demonstrate the ability of Tri-DAP to enhance TLR agonist induced pro-inflammatory cytokines in a MAPK dependent manner, but that ERK MAPK may have a negative influence on Tri-DAP-induced IL-27 production.

### Example 8

### Treatment with TriDAP Attenuates EAE in the SJL Mouse Relapsing-Remitting EAE Model

This experiment considered whether treatment with the Nod-1 agonist TriDAP attenuated EAE in the SJL mouse relapsing-remitting EAE model.

### Materials and Methods:

EAE (experimental autoimmune encephalomyelitis) was induced in SJL mice by immunization with protein lipid protein (PLP) in complete Freund's adjuvant (CFA) followed by Bordetella pertussis toxin (PT). SJL mice were developed by James Lambert at The Jackson Laboratory in 1955 from three different sources of Swiss Webster mice. The strain is characterized by its susceptibility to experimental autoimmune encephalomyelitis (EAE) for multiple sclerosis research, and therefore provides an improved model (over the MOG (myelin oligodendrocyte glycoprotein) model) for assessing the therapeutic potential of compounds. Mice were treated with PBS (control) or TriDAP (100 μg/mouse) every 2 days from day 12. Clinical scores were assessed daily and body weights were recorded. Disease severity was graded as follows: grade 0: normal, grade 1: limp tail, grade 2: wobbly gait; grade 3: hind limb weakness; grade 4: hind limb paralysis; grade 5: tetraparalysis/death. † indicates control mice were sacrificed due to severity of EAE disease.

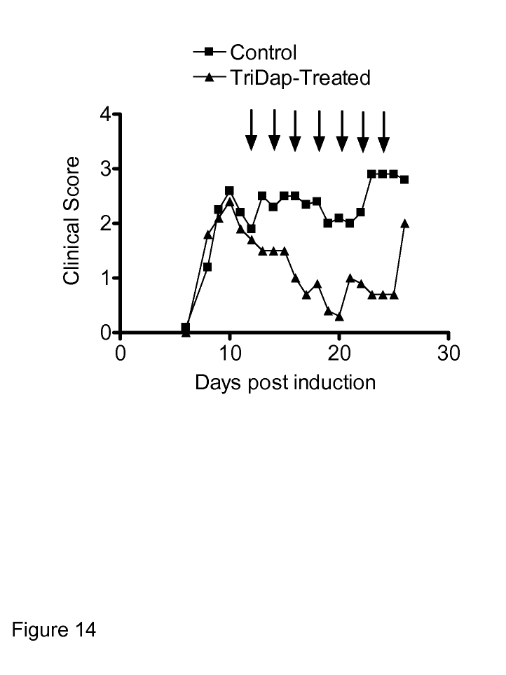

The results are shown in FIG. 14. The results are mean clinical scores for 5-6 mice per group. The results show a reduction in clinical scores for the TriDAP treated animals.

### Example 9

### Effect of TriDAP Treatment on PLP-Specific IL-17 and IFN-γ in the Relapsing-Remitting EAE Model

This experiment considers the effect of treatment with the Nod-1 agonist, TriDAP on the expression of PLP-specific IL-17 and IFN-γ in the relapsing-remitting EAE model.

### Materials and Methods

EAE was induced in SJL mice by immunization with protein lipid protein (PLP) in complete Freund's adjuvant (CFA) followed by pertussis toxin (PT). Mice were treated with PBS (control) or TriDAP (100 μg/mouse) every 2 days from day 12. Mice were sacrificed on day 26, and lymph node or spleen cells restimulated in vitro with PLP (1-25 μg/ml). After 3 days, IL-17 and IFNγ production in supernatants were quantified by ELISA.

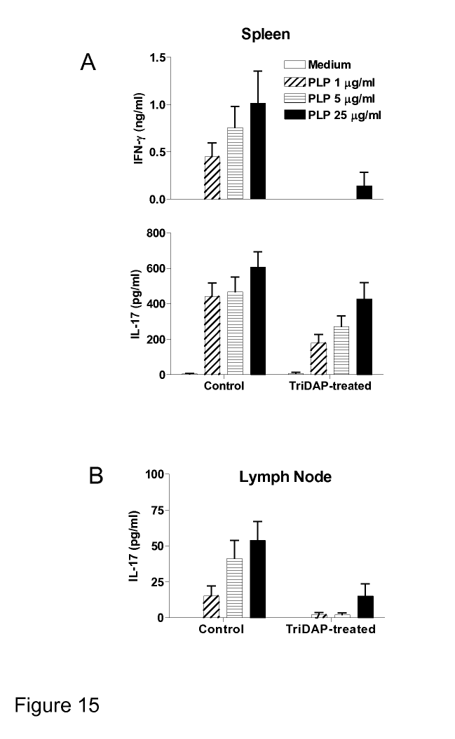

The results are shown in FIG. 15. FIG. 15A shows IFN-gamma and IL-17 expression in spleen cells. FIG. 15B shows IL-17 production in the lymph node. These results show that there is an on-going reduction of IFN-gamma and IL-17 levels following Tri-DAP treatment.

### Example 10

### Expression of TLR Agonist Induced IL-27, IL-10 and TGF-Beta in Mice Treated with TriDAP

This experiment shows that levels of TLR-agonist induced IL-27, IL-10 TGF-β levels are enhanced by peritoneal exudate cells from mice treated with TriDAP

### Materials and Methods

Mice were treated with either PBS or TriDAP (100 μg/mouse/day i.p.) for 5 days. Peritoneal exudate cells (PECs; 1×10⁶/ml) were stimulated with medium alone (control), or with a Toll-like Receptor agonist selected from the following: the TLR4 agonist LPS (lipopolysaccharide) (100 ng/ml), the TLR9 agonist CpG (5 μg/ml), or the TL2 agonist Pam3Csk (500 ng/ml). Supernatants were collected after 24 hours and cytokine concentrations were quantified by ELISA.

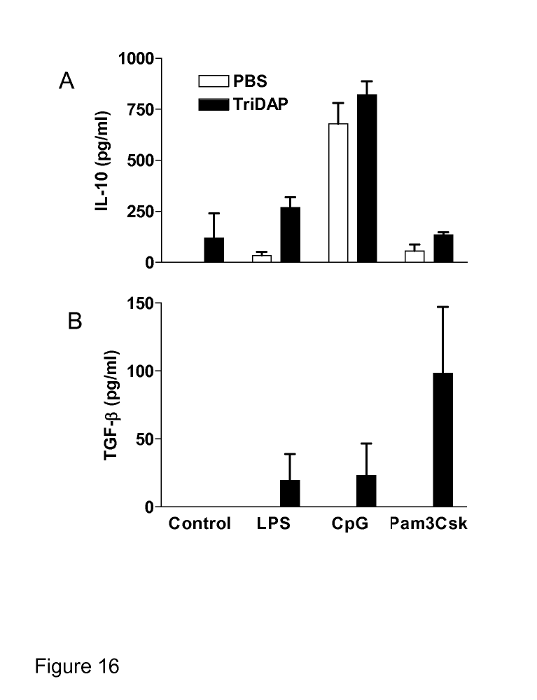

The results are shown in FIG. 16. FIG. 16A shows IL-10 expression in the supernatants of mouse treated with and without TriDAP, while FIG. 16B shows TGF-beta expression. These results show that PEC from mice treated in vivo with TriDAP secrete higher concentrations of the immunosuppressive cytokines, IL-10 and TGF-beta following re-stimulation in vitro with TLR agonists.

### Example 11

### TLR-Induced IL-10 and TGF-β by Spleen Cells from Mice Treated with TriDAP

This experiment measured IL-10 and TGF-β expressed by spleen cells derived from TriDAP treated mice.

Mice treated with either PBS or TriDAP (100 μg/mouse/day i.p.) for 5 days. Spleen cells (PECs; 1×10⁶/ml) were stimulated with medium alone (control), or with a Toll-like Receptor agonist selected from the following: the TLR4 agonist LPS (lipopolysaccharide) (100 ng/ml), the TLR9 agonist CpG (5 μg/ml), or the TL2 agonist Pam3Csk (500 ng/ml). Supernatants were collected after 24 hours and cytokine concentrations were quantified by ELISA.

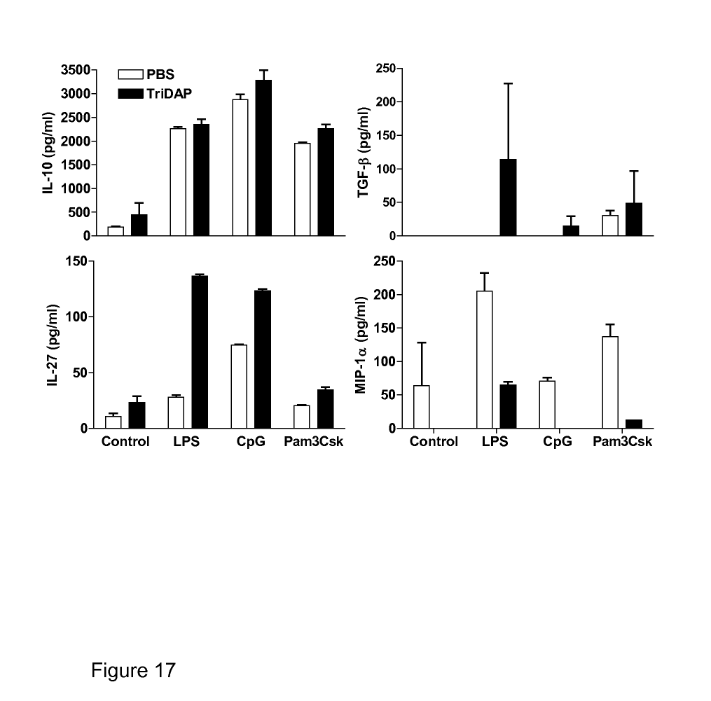

Results are shown in FIG. 17. FIG. 17 shows 4 graphs representing the expression levels of the cytokines IL-10, IL-27 and TGF-β and of the chemokine MIP-1α in the supernatants of mice treated with PBS or TriDAP along with a TLR agonist. Expression levels of IL-10 and IL-27 are seen to be enhanced where the spleen cells were stimulated with a TLR agonist. These results show that spleen cells from mice treated in vivo with TriDAP secrete higher concentrations of the immunosuppressive cytokines, IL-10, IL-27 and TGF-beta following re-stimulation in vitro with TLR agonists.

### Example 12

### Effect of TriDAP on Antigen Presenting Cells

In this experiment, the ability of antigen presenting cells (APC) to activate memory T cells in mice treated with TriDAP is assessed.

### Materials and Methods

Mice were treated with either PBS or TriDAP (100 μg/mouse/day i.p.) for 5 days. Peritoneal exudate stimulator cells (PECs) were irradiated and used as antigen presenting cells (APC) for MOG-specific T cells. T cells from mice immunized with MOG (Myelin oligodendrocyte glycoprotein) and CFA (1×10⁶/ml) were culture with PECs (1×10⁶/ml) and MOG (20 μg/ml). Supernatants were collected after 72 hours and IFN-γ concentrations were quantified by ELISA.

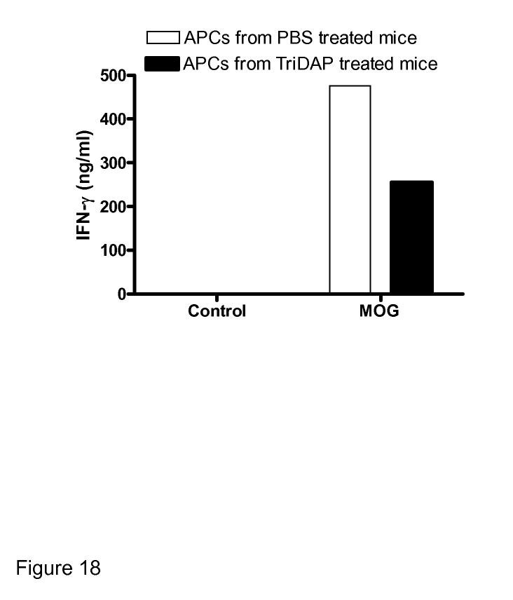

The results are shown in FIG. 18 where it can be seen that antigen-specific T cells secrete less IFN-gamma when stimulated with antigen presenting cells obtained from TriDAP treated when compared with control (PBS) treated mice. This indicates that antigen presenting cells isolated from TriDAP treated mice are less effective at inducing T cell activation.

### Example 13

### TriDAP Suppresses the Ability of TLR Agonist-Activated Dendritic Cells to Activate Th17 Cells In Vitro

CD4 T cells from mice immunized with MOG and LPS, were cultured in vitro with dendritic cells stimulated with LPS (10 or 100 ng/ml) with and without TriDAP (100 μg/ml) in the presence of KLH (20 μg/ml). Supernatants were collected after 72 hours and IL-17 concentrations were quantified by ELISA. *P<0.05, ***P<0.001, with and without TriDAP.

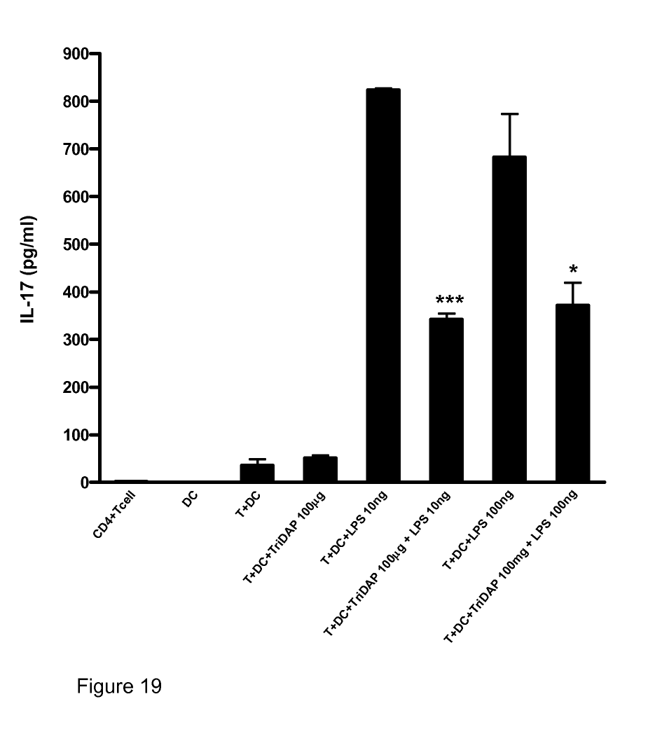

The results are shown in FIG. 19, which shows a graph showing IL-17 expression levels by antigen-specific T cells stimulated with dendritic cells pulsed with antigen and LPS with and without TriDAP. The results show that the treatment of dendritic cells with TriDAP results in a significant reduction in their ability to activate IL-17 production by CD4 T cells.

### Example 14

### TriDap Induces IL-10 and IL-27 and Enhances TLR-Induced IL-10 and IL-27 Production by Human Dendritic Cells

Dendritic cells cultured from human PBMC with IL-4 and GM-CSF were stimulated with TriDap (100 μg/ml), LPS (10 or 100 ng/ml), LPS and TriDAP or medium only. Supernatants were collected after 24 hours and IL-27 and IL-10 concentrations were quantified by ELISA. **P<0.01, ***P<0.001, with versus without TriDAP.

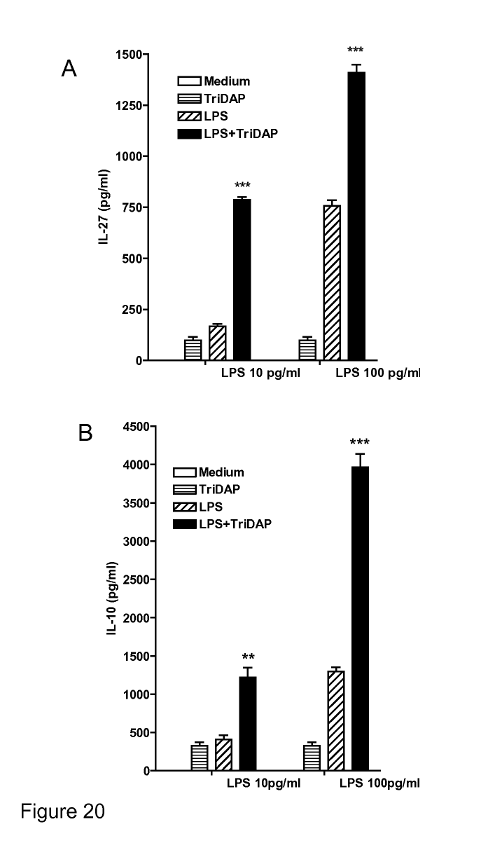

The results are shown in FIG. 20. FIG. 20A shows IL-27 expression levels in human derived dendritic cells stimulated with TriDAP, while FIG. 20B shows IL-10 expression levels in the same cell population. The results show that human derived dendritic cells express IL-27, following in vitro stimulation with TriDAP, this being shown by measurement of IL-27 protein levels. Furthermore, IL-27 expression by human dendritic cells is shown, this being consistent with the expression of IL-27 by murine derived dendritic cells, as shown by previous examples. Lastly, the results show that IL-27 can be expressed by human dendritic cells without the need for co-stimulation with a Toll-like Receptor agonist. However, the expression of IL-27 can be significant enhanced by co-stimulation of an antigen presenting cell with at least one Toll-like Receptor agonist compound, such as lipopolysaccharide Accordingly, it is shown that TriDAP acts to independently induce IL-27 production by dendritic cells and does not just merely function to modulate TLR-agonist driven IL-27 production in dendritic cells.

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention. Reference to any prior art in this specification is not, and should not be taken as, an acknowledgment or any form of suggestion that this prior art forms part of the common general knowledge in any country.

1. A method for treating and/or preventing an autoimmune disease or chronic inflammatory disease which is mediated by autoreactive T-helper 1 lymphocytes (Th1 T-cells) and/or T-helper 17 lymphocytes (Th17 T cells), the method comprising the steps of:
providing a therapeutically effective amount of a composition which comprises a diaminopimelic acid (DAP)-containing muropeptide compound, and
administering said composition to a subject in need of such treatment in an amount sufficient to suppress the activation of Th 17 T cells and/or Th 1 T cells.

2. The method as claimed in claim 1 wherein the diaminopimelic acid (DAP)-containing muropeptide compound is TriDAP.

3. The method as claimed in claim 1 wherein the autoimmune disease is selected from the group consisting of multiple sclerosis (MS), rheumatoid arthritis (RA) and type 1 diabetes, or wherein the chronic inflammatory disease is selected from the group consisting of inflammatory bowel disease (IBD), Crohn's disease, ulcerative colitis and psoriasis.

4. The method as claimed in claim 1 wherein the method further comprises the step of administering at least one Toll-like Receptor agonist to the subject.

5. The method as claimed in claim 4 wherein the at least one Toll-like Receptor agonist is an agonist for at least one of Toll-like Receptor 2, Toll-like Receptor 4 or Toll-like Receptor 9.

6. The method as claimed in claim 4 the Toll-like Receptor agonist is selected from the group consisting of LPS (lipopolysaccharide), CpG motifs, CpG-containing oligodeoxynucleotides (CpG ODN), dsRNA, Poly (I:C) and Pam-3Cys.

7. The method as claimed in claim 1, wherein the method further comprises the step of administering at least one ERK protein kinase inhibitor to the subject.

8. The method as claimed in claim 7 wherein the at least one ERK protein kinase inhibitor is PD98059 or U0126.

9. A pharmaceutical composition for use in the treatment and/or prevention of an autoimmune disease or a chronic inflammatory condition which is mediated by autoreactive Th1 and/or Th17 T cells, the composition comprising a diaminopimelic acid (DAP)-containing muropeptide compound along with at least one pharmaceutical excipient, diluent or carrier.

10. The pharmaceutical composition as claimed in claim 9 wherein the diaminopimelic acid (DAP)-containing muropeptide is TriDAP.

11. The pharmaceutical composition as claimed in claim 9 wherein the autoimmune disease is selected from: multiple sclerosis (MS), rheumatoid arthritis (RA) and type 1 diabetes, or wherein the chronic inflammatory disease is selected from the group consisting of inflammatory bowel disease (IBD), Crohn's disease, ulcerative colitis and psoriasis.

12. The pharmaceutical composition as claimed in claim 9 wherein the composition further comprises at least one Toll-like Receptor agonist.

13. The pharmaceutical composition as claimed in claim 12 wherein the at least one Toll-like Receptor agonist is an agonist for at least one of Toll-like Receptor 2, Toll-like Receptor 4 or Toll-like Receptor 9.

14. The pharmaceutical composition as claimed in claim 12 wherein the Toll-like Receptor agonist is selected from the group consisting of LPS (lipopolysaccharide), CpG motifs, CpG-containing oligodeoxynucleotides (CpG ODN), dsRNA, Poly (I:C) and Pam-3Cys.

15. The pharmaceutical composition as claimed in claim 9, wherein the composition further comprises at least one ERK protein kinase inhibitor.

16. The pharmaceutical composition as claimed in claim 15 wherein the at least one ERK protein kinase inhibitor is PD98059 or U0126.

17-32. (canceled)

33. A method for treating multiple sclerosis, the method comprising the steps of:
providing a NOD-1 agonist which suppresses a T-helper 17 lymphocyte (Th17) mediated immune response and/or a T-helper 1 lymphocyte (Th1) mediated immune response, which upregulates production of the cytokine IL-27 and downregulates production of the cytokines TNF-γ, TNF-α, IL-17 and IL-23; and
administering said NOD-1 agonist to a subject in need of such treatment.
